(19) United States
(12) Patent Application Publication
(10) Pub. No.: US 2017/0290608 A1
(43) Pub. Date: Oct. 12, 2017

(54) SPINAL FIXATION SYSTEMS AND METHODS

(71) Applicant: Spinal USA, Inc., Parsippany, NJ (US)

(72) Inventors: Robert Neal, Morris Plains, NJ (US); Scott Mozeleski, Concord, NC (US); Michael Campbell, Florham Park, NJ (US)

(21) Appl. No.: 15/411,145

(22) Filed: Jan. 20, 2017

Related U.S. Application Data
(60) Provisional application No. 62/286,240, filed on Jan. 22, 2016.

(51) Int. Cl. A61B 17/70

(52) U.S. Cl. CPC A61B 17/7037; A61B 17/70; A61B 17/7034

(57) ABSTRACT

Translation screw assemblies that can include a screw, housing, load plate, and collet are described herein. The translation screw assembly advantageously allows for relative translation, angulation or pivoting, and/or rotation between the screw and the housing. Occipital plate assemblies are also described herein. An occipital plate assembly can include a plate body and one or more rod receiving assemblies. The plate assembly can include an occipital protuberance tab that can be bent relative to or sheared off from the plate body. The rod receiving assembly can include a pivot post, housing, clamp plate, and retaining washer. The rod-receiving assemblies advantageously allow for relative medial-lateral translation, medial-lateral angulation, and/or cranial-caudal angulation between the rod-receiving assemblies or portions thereof (e.g., the housings) and plate body.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. The present application claims priority to U.S. Provisional Application No. 62/286,240, filed Jan. 22, 2016, the entirety of which is hereby incorporated by reference herein.

### BACKGROUND

### Field

The present disclosure generally relates to systems and methods for spinal fixation. More particularly, the present disclosure relates to plates for fixation to a portion of a patient's skull, such as the occipital bone, and screw assemblies that allow for translational movement.

### Description of the Related Art

Spinal fusion encompasses a surgical technique in which two or more vertebrae are connected together. This technique may be used for multiple indications, including abnormal spinal curvature (e.g., scoliosis) and weakening or injuring of the vertebrae or spinal disc.

In some instances, this process is accomplished and/or supplemented using a plurality of screws implanted into adjacent vertebrae and joined together by a series of one or more rods. The screw may have an enlarged head that interfaces with a housing having a corresponding cavity, thus allowing for a range of articulation between the screw and the housing. After the screw is implanted into bone, a rod may be placed in the housing, and a set screw may be delivered into engagement with the housing, applying a downward force on the rod to hold the assembly together.

In some instances, spinal fusion is accomplished and/or supplemented using a plate to join together adjacent vertebrae or other bones and/or to anchor various components to various bones. The plate is affixed by implanting a plurality of screws through the plate and into the bone(s).

In some cases, a plate is attached to the patient's occipital bone, and a plurality of screws are implanted in adjacent cervical vertebrae. The plate and screws can be joined together with one or more rods to help stabilize and/or promote fusion of the junction between the occipital bone and cervical spine. The rods can be curved or have curved portions to accommodate the curvature of the junction.

### SUMMARY

In some embodiments, a translation screw assembly includes a screw and a housing. The screw has a threaded shaft and an enlarged head at a proximal end. The housing has an upper portion with an upper opening and a lower portion with a lower opening extending along a first axis of the housing. The enlarged head of the screw is disposed within the housing and the shaft extends out of the housing through the lower opening. The housing also has a third opening and a fourth opening along a second axis that is transverse to the first axis and adapted to receive an elongated rod. The screw is configured to translate, pivot, and rotate relative to the housing.

The translation screw assembly can further include a generally circular collet disposed around the enlarged head of the screw and disposed adjacent the lower opening of the housing, and the screw is configured to rotate and pivot relative to the collet. In some such embodiments, a lower portion of the housing is oblong and inner surfaces of sides of the lower portion of the housing adjacent the lower opening are straight, an outer surface of the collet comprises two opposing straight portions, and the straight portions of the outer surface of the collet are configured to contact and translate along the straight inner surfaces of the sides of the lower portion of the housing such that the collet and screw can translate relative to the housing and the collet is rotationally fixed relative to the housing. The translation screw assembly can further include a load plate disposed within the housing, wherein at least a portion of the screw head is configured to contact an inner surface of a lower portion of the load plate. In some embodiments, the housing includes an upper component and a lower component, the upper component comprising the upper portion and the upper opening and the lower component comprising the lower portion and the lower opening, wherein the upper component and lower component are fixedly coupled together.

In some embodiments, an occipital plate assembly includes a plate body and an occipital protuberance tab coupled to the plate body via at least one junction. The plate body includes at least one screw receiving hole configured to receive a bone screw therethrough. A top surface of the at least one junction includes a material relief comprising a reduced thickness area configured such that if a sufficient downward force is applied to the occipital protuberance tab, the occipital protuberance tab shears off from the plate body at the at least one junction.

In some embodiments, a bottom surface of the at least one junction comprises a second material relief comprising a reduced thickness area configured such that the occipital protuberance tab can be bent upwards relative to the plate body. In some such embodiments, the second material relief is wider than the material relief. The occipital protuberance tab can include a screw receiving hole configured to receive a bone screw therethrough. In some embodiments, the occipital plate assembly further includes at least one rod receiving assembly configured to receive a spinal rod.

In some embodiments, an occipital plate assembly includes a plate body and at least one rod receiving assembly coupled to the plate body. The plate body includes at least one screw receiving hole configured to receive a bone screw therethrough. The rod receiving assembly includes a housing configured to receive an elongated rod, wherein the housing is configured to translate in a medial-lateral direction, angulate in a medial-lateral direction, and angulate in a cranial-caudal direction relative to the plate body.

In some embodiments, the rod receiving assembly further includes a pivot post, a clamp plate, and a retaining washer. The housing has an upper portion with an upper opening and a lower portion with a lower opening extending along a first axis of the housing, an enlarged head of the pivot post is disposed within the housing and a shaft of the pivot post extends out of the housing through the lower opening, and the housing has a third opening and a fourth opening along a second axis transverse to the first axis adapted to receive the elongated rod. The plate body includes a mounting portion having a first slot therethrough. The clamp plate includes a recess and a second slot therethrough. The housing is partially seated in the recess of the clamp plate and the clamp plate is disposed on the mounting portion. The shaft of the pivot post extends through the second slot of the clamp plate and the first slot of the plate body and is secured by the retaining washer disposed adjacent a bottom surface of the mounting portion.

In some embodiments, the housing, pivot post, clamp plate, and retaining washer are configured to translate along the mounting portion. In some embodiments, the second slot of the clamp plate comprises a central portion through which the shaft of the pivot post extends and two side portions extend from opposites sides of the central portion, the housing comprises two tabs protruding from a bottom surface of the housing on opposite sides of the lower opening, and the tabs extend into the side portions of the second slot of the clamp plate and are configured to angulate relative to the clamp plate within the side portions of the second slot. In some embodiments, a bottom surface of the clamp plate comprises a curved ridge, a top surface of the mounting portion comprises a curved channel, the ridge is at least partially disposed in the channel, and the ridge is configured to rock within the channel to allow for cranial-caudal angulation of the housing relative to the plate body.

In some embodiments, an occipital fixation system includes an occipital plate assembly, a plurality of screw assemblies configured to be inserted into adjacent vertebrae of a patient's cervical spine, and a pair of rods. The occipital plate assembly includes a plate body sized for positioning on a patient's occipital bone, the plate body comprising at least one screw receiving hole configured to receive a bone screw therethrough, and a pair of rod receiving assemblies coupled to the plate body. Each rod receiving assembly includes a housing configured to receive an elongated rod, and each housing is configured to translate in a medial-lateral direction, angulate in a medial-lateral direction, and angulate in a cranial-caudal direction relative to the plate body. Each of the screw assemblies includes a screw having a threaded shaft and an enlarged head at a proximal end and a housing. The housing has an upper portion with an upper opening and a lower portion with a lower opening extending along a first axis of the housing, the enlarged head of the screw is disposed within the housing and the shaft extends out of the housing through the lower opening, the housing has a third opening and a fourth opening along a second axis transverse to the first axis adapted to receive an elongated rod, and the screw is configured to translate, pivot, and rotate relative to the housing. The pair of rods are sized to be received within the rod receiving assemblies of the occipital plate assembly and the housings of the screw assemblies to connect the occipital plate assembly when positioned on the patient's occipital bone to the screw assemblies when inserted into adjacent vertebrae of the patient's cervical spine.

All of these embodiments are intended to be within the scope of the disclosure herein. These and other embodiments will become readily apparent to those skilled in the art from the following detailed description having reference to the attached figures, the disclosure not being limited to any particular disclosed embodiment(s).

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 shows a perspective view of an example embodiment of a translation screw assembly.

FIG. 2 shows a sectional perspective view of the translation screw assembly of FIG. 1.

FIG. 3 shows an exploded view of the translation screw assembly of FIG. 1.

FIG. 4 shows a side view of the translation screw assembly of FIG. 1.

FIG. 5 shows a sectional side view of the translation screw assembly of FIG. 1.

FIG. 6 shows a front view of the translation screw assembly of FIG. 1.

FIG. 7 shows a sectional front view of the translation screw assembly of FIG. 1.

FIG. 8 shows a sectional perspective view of the translation screw assembly of FIG. 1 with a screw of the assembly translated relative to a housing of the assembly.

FIG. 9 shows a bottom perspective view of the translation screw assembly of FIG. 1.

FIG. 10 shows a bottom view of the translation screw assembly of FIG. 1.

FIG. 11 shows a top view of the translation screw assembly of FIG. 1.

FIGS. 12 and 13 show sectional side views of the translation screw assembly of FIG. 1 showing angulation of the screw relative to the housing.

FIG. 14A shows a bottom perspective view of a collet of the translation screw assembly of FIG. 1.

FIG. 14B shows a bottom view of the collet of FIG. 14A.

FIG. 15 shows a sectional view of the collet of FIG. 14A.

FIG. 16 shows a bottom perspective view of an upper component of the housing of the translation screw assembly of FIG. 1.

FIG. 17 shows a front perspective view of a load plate of the translation screw assembly of FIG. 1.

FIG. 18 shows a sectional perspective view the load plate of FIG. 17.

FIG. 19 shows a perspective view of an example embodiment of an occipital plate assembly.

FIG. 20 shows a front view of the occipital plate assembly of FIG. 19.

FIG. 21 shows a side view of the occipital plate assembly of FIG. 19.

FIG. 22 shows a top view of the occipital plate assembly of FIG. 19.

FIG. 23 shows a sectional view of the occipital plate assembly of FIG. 19 taken along line 23-23 shown in FIG. 22.

FIG. 24 shows an exploded view of the occipital plate assembly of FIG. 19.

FIGS. 25A-25C show sectional front views of the occipital plate assembly of FIG. 19 showing medial-lateral translation of rod receiving assemblies relative to a plate body.

FIGS. 26A-26B show top views of the occipital plate assembly of FIG. 19 showing medial-lateral angulation of housings of the rod receiving assemblies relative to the plate body.

FIGS. 27A-27C show sectional and side views of the occipital plate assembly of FIG. 19 showing cranial-caudal angulation of the housings relative to the plate body.

FIG. 28A shows a top view of a clamp plate of the rod receiving assembly of the occipital plate assembly of FIG. 19.

FIG. 28B shows a side view of the clamp plate of FIG. 28A.

FIG. 28C shows a bottom perspective view of the clamp plate of FIG. 28A.

FIG. 29A shows a bottom view of the housing of the rod receiving assembly of the occipital plate assembly of FIG. 19.

FIG. 29B shows a side view of the housing of FIG. 29A.

FIG. 29C shows a bottom perspective view of the housing of FIG. 29A.

FIG. 30A shows a perspective view of another example embodiment of an occipital plate assembly.

FIG. 30B shows a bottom perspective view of the occipital plate assembly of FIG. 30A.

FIG. 31A shows a top view of another example embodiment of an occipital plate assembly.

FIG. 31B shows a bottom view of the occipital plate assembly of FIG. 31A;

FIG. 31C shows another example embodiment of an occipital plate assembly.

FIG. 31D shows another example embodiment of an occipital plate assembly.

FIG. 32A shows a rear view of the skull and a portion of the spine.

FIG. 32B shows an example embodiment of the occipital plate assembly of FIG. 31A, rods, and screw assemblies secured to a skull and spine.

FIG. 33 shows an exploded view of an example embodiment of a screw assembly including a screw, housing, c-clip ring, and saddle.

FIG. 34 shows an assembled cross-sectional view of the screw assembly of FIG. 33.

FIG. 35 shows a cross-sectional view of the housing of the screw assembly of FIGS. 33-34.

FIGS. 36A-36B shows perspective views of the c-clip ring of the screw assembly of FIGS. 33-34.

FIG. 36C shows a cross-sectional view of the c-clip ring of FIGS. 36A-36B.

FIGS. 37A and 37B show example embodiments of pre-lordosed rods.

FIG. 37C shows an example embodiment of an adjustable hinged rod.

FIG. 37D shows a sectional view of the adjustment mechanism of the hinged rod of FIG. 37C.

FIGS. 38A-38D show example embodiments of occipital screws.

FIG. 39 shows an example embodiment of a kit or implant tray including occipital plate assemblies, occipital screws, and rods.

### DETAILED DESCRIPTION

Although certain embodiments and examples are described below, those of skill in the art will appreciate that the disclosure extends beyond the specifically disclosed embodiments and/or uses and obvious modifications and equivalents thereof. Thus, it is intended that the scope of the disclosure herein disclosed should not be limited by any particular embodiments described below.

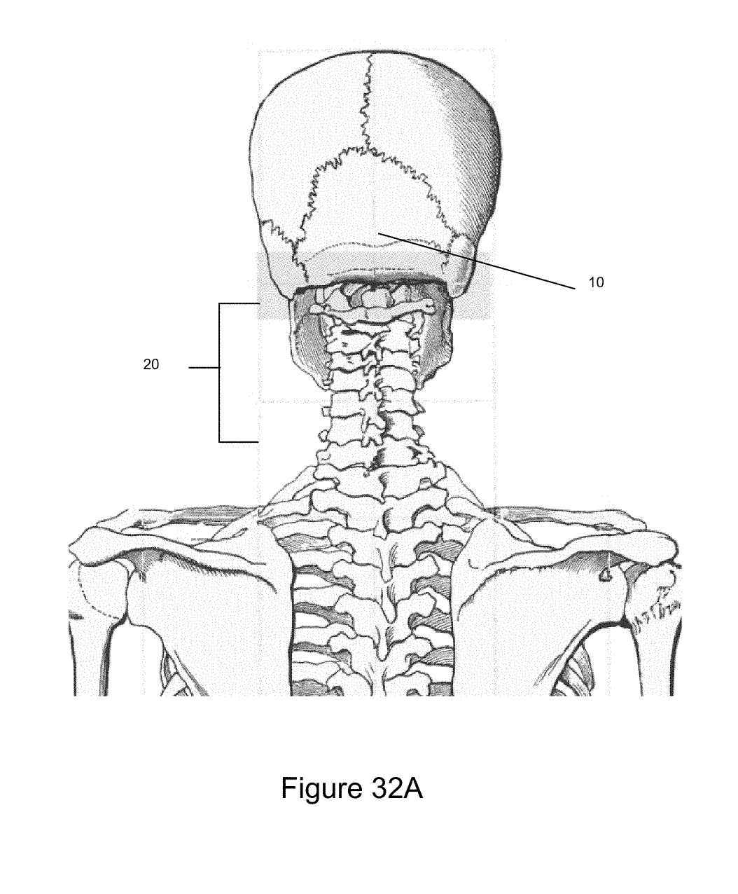

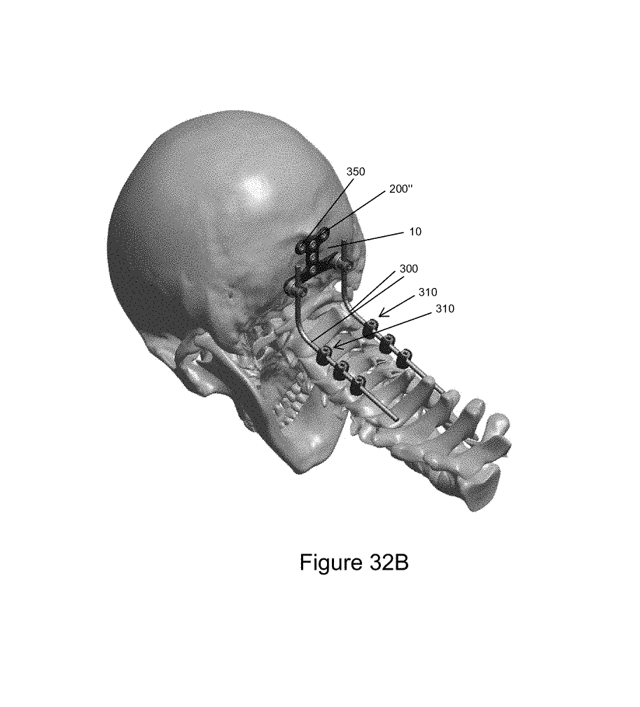

Certain embodiments of the present disclosure relate to an occipital plate system that is secured to a patient's occipital bone 10, shown in FIG. 32A, and one or more vertebrae in the cervical spine 20. The system can include an occipital plate that is fixed to the patient's occipital bone 10, one or more screws that are fixed to posterior aspects of one or more cervical vertebrae, and one or more rods that connect the plate with one or more of the screws, for example as shown in FIG. 32B. In some embodiments, the screws are translation screw assemblies as described herein. In some embodiments, the screws are friction screw assemblies as described herein. In some embodiments, translation screw assemblies and/or friction screw assemblies as described herein can be used independently from an occipital plate, with other spinal implants, and/or in other regions (e.g., thoracic or lumbar) of the spine. In some embodiments, an occipital plate and/or one or more translation screw assemblies and/or friction screw assemblies as described herein can be used in combination with cross-connectors, for example, rod-to-rod cross-connectors (i.e., connectors that extend between and connect two rods) or head-to-head cross-connectors (i.e., connectors that extend between and connect housings of screw assemblies), one or more lateral offsets, and/or other components and/or instruments.

### Translation Screw Assembly

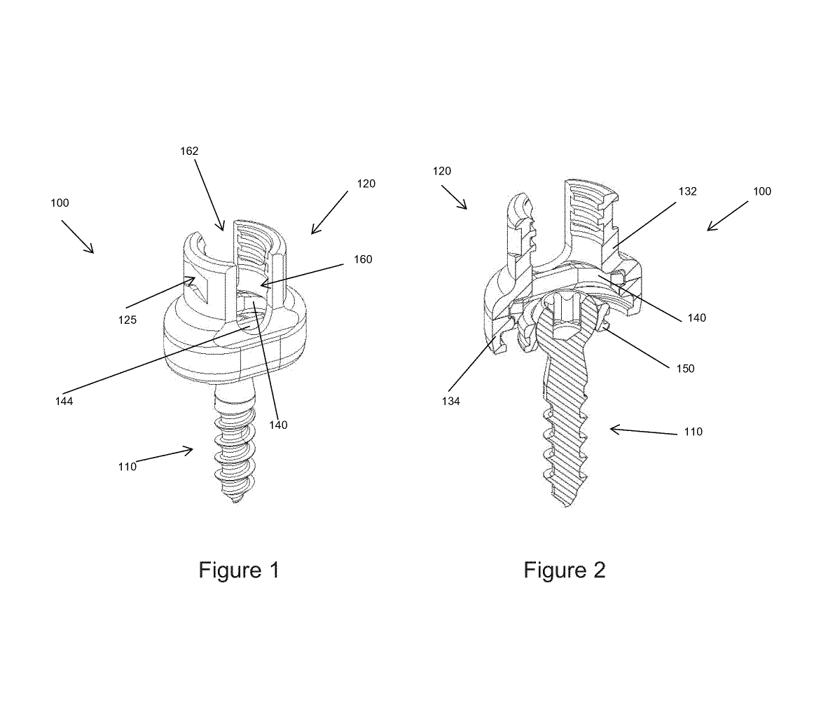

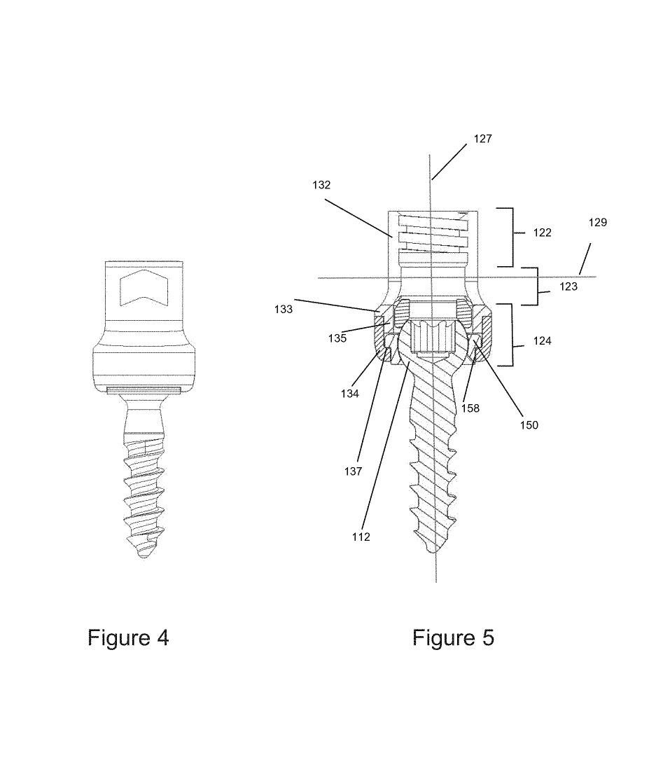

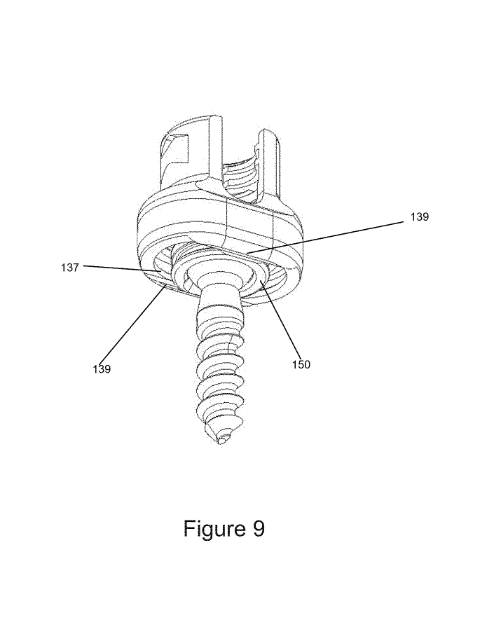

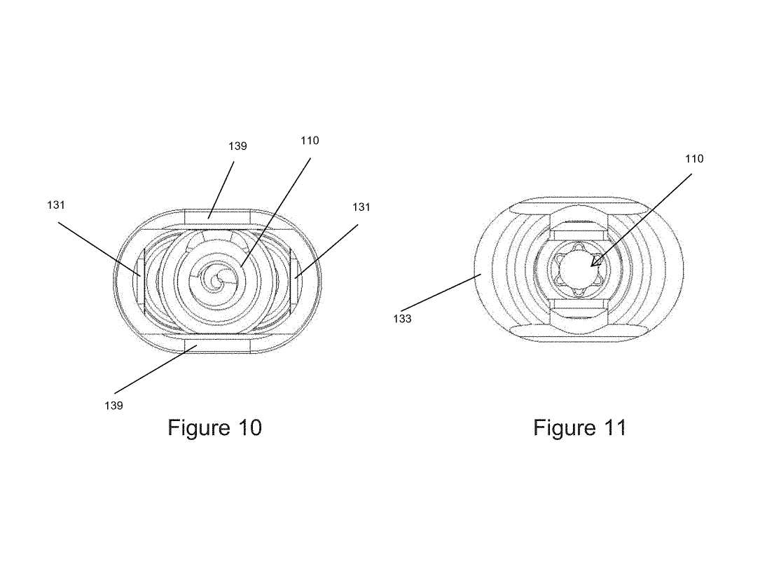

FIGS. 1-13 illustrate an example embodiment of a translation screw assembly 100. The translation screw assembly 100 in some embodiments may be used to attach to vertebrae on the posterior side of the cervical spine, e.g., to the pedicle, and may further be used to receive a rod that will connect to other screws or other devices attached to vertebrae of the cervical spine, the thoracic spine, and/or the occipital bone. Multiple translation screw assemblies 100 may be provided in use, for example, in adjacent vertebral bodies and on opposite sides of a patient's midline. As shown, the translation screw assembly 100 can include a screw 110 configured to be secured to a portion of a patient's spine, a housing 120, a load plate 140, and a collet 150. The translation screw assembly 100 can further include a set screw and a spinal rod (not shown). In some embodiments, one or more of the components described herein is made of a metal, such as titanium or alloys thereof, Cobalt-Chromium (CoCr), and/or any other implantable grade material. For example, one or more components can be made at least partially of titanium 6AL 4V ELI.

The screw 110 has an enlarged head 112 at a proximal end and a shaft or body portion 114 extending from the head 112 to a tip at a distal end. The head 112 can be approximately spherical or ball-shaped. In the illustrated embodiment, the head 112 is partially spherical and has a flattened proximal end or surface that can receive a screwdriver. The shaft 114 can be at least partially threaded and adapted to be implanted into a patient's spine, for example, into the pedicle of a vertebra. In the illustrated embodiment, the screw 110 is self-tapping and is not cannulated. However, in other embodiments, the screw 110 may be non-self-tapping and/or cannulated.

As shown in FIGS. 5 and 7, the housing 120 includes an upper portion 122 having an upper opening 126, a lower portion 124 having a lower opening 128, and an intermediate portion 123. The upper opening 126 and lower opening 128 can extend along a first axis 127 of the housing 120. The upper opening 126 and lower opening 128 can be connected so as to create a through hole passing from the upper opening 126, through the upper portion 126, intermediate portion 123, and lower portion 124, to the lower opening 128. In use, the screw 110 is disposed within the housing 120 such that the head 112 is within the lower portion 124 and the shaft 114 extends through the lower opening 128. A diameter of the lower opening 128 can be greater than a diameter of the upper opening 126. A diameter of the enlarged head 112 of the screw 110 can be smaller than the diameter of the upper opening 126 and the diameter of the lower opening 128.

The housing 120 further includes a third opening 160 and a fourth opening 162 (shown in FIG. 1) extending along a second axis 129 of the housing 120 that is transverse to the first axis 127. The third opening 160 and fourth opening 162 intersect an upper edge of the housing 120 and separate the upper portion 122 and intermediate portion 123 of the housing 120 into two opposing arms. In the illustrated embodiment, the third opening 160 and fourth opening 162 are generally U-shaped, although other shapes are also possible. In use, the third opening 160 and fourth opening 162 receive the rod such that the rod is disposed within the intermediate portion 123, and lower or distal portions of the third opening 160 and fourth opening 162 define a seat for the rod.

In the illustrated embodiment, an interior of the upper portion 122 is generally cylindrical. An exterior of the upper portion 122 can also be generally cylindrical. In other embodiments, the exterior of the upper portion 122 can have a squared or slightly squared shape. In the illustrated embodiment, an interior and an exterior of the lower portion 124 are generally oblong along a third axis 136 of the housing 120 that is transverse to the first 127 and second 129 axes.

In the illustrated embodiment, the upper portion 122 of the housing 120 is internally threaded to receive and engage an externally threaded set screw. The threading may not extend below a point at or below the rod when the rod is disposed in the housing 120 in use. In other embodiments, the upper portion 122 may be externally threaded to receive and engage an internally threaded set screw or cap, or the upper portion 122 may receive and engage a closure mechanism via means other than threading. The set screw can have square or modified square threads, although other types of threads are also possible. An outer surface of the housing 120 can include one or more indentations 125 that receive an insertion tool during use.

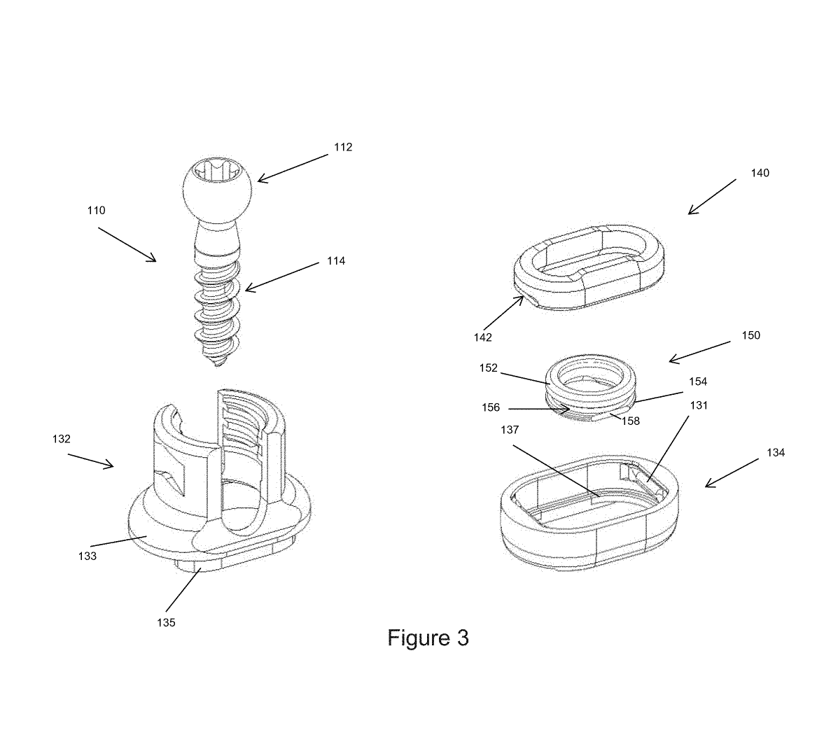

In the illustrated embodiment, and with reference to FIG. 3, the housing 120 includes an upper component 132 and a lower component 134. The upper 132 and lower 134 components are coupled together, either removably or permanently. For example, in the illustrated embodiment, the upper 132 and lower 134 components are welded together. The upper 132 and lower 134 components can be fixedly coupled such that the upper 132 and lower 134 components are immovable relative to each other. In some alternative embodiments, the housing 120 is a unitary, integrally formed component.

As shown, the upper component 132 can include or form the upper portion 122 and intermediate portion 123 of the housing 120. In the illustrated embodiment, the upper component 132 includes a flange 133 and a lower extension 135 (as shown in, for example, FIGS. 3 and 5). The flange 133 can be positioned below the intermediate portion 123 of the housing. The lower extension 135 extends downward or distally from a lower or distal surface of the flange 133. The lower extension 135 can extend around the entire lower opening 128 or may be discontinuous. As shown in FIG. 16, in the illustrated embodiment, the lower extension 135 includes gaps 135a along the ends of the upper component 132 (where ends are generally opposite each other along third axis 136 and/or relatively more parallel to second axis 129, and sides extend generally opposite each other along an axis parallel to second axis 129 and/or relatively more parallel to third axis 136).

The flange 133 has a greater length (along a direction parallel to third axis 136) and width (along a direction parallel to second axis 129) than the upper portion 122 of the housing 120 and the lower extension 135. The lower portion 124 of the housing 120 can include the flange 133 and the lower extension 135 of the upper component 132 and the lower component 134. As shown, the flange 133, lower extension 135, and/or lower component 134 can be generally oblong. As shown in FIG. 5, when the upper 132 and lower 134 components are assembled, an inner surface of an upper portion of the lower component 134 contacts an outer surface of the lower extension 135, and an upper surface or edge of the lower component 134 contacts a lower surface of the flange 133. The upper 132 and lower 134 components can be welded or otherwise secured together along these contact points.

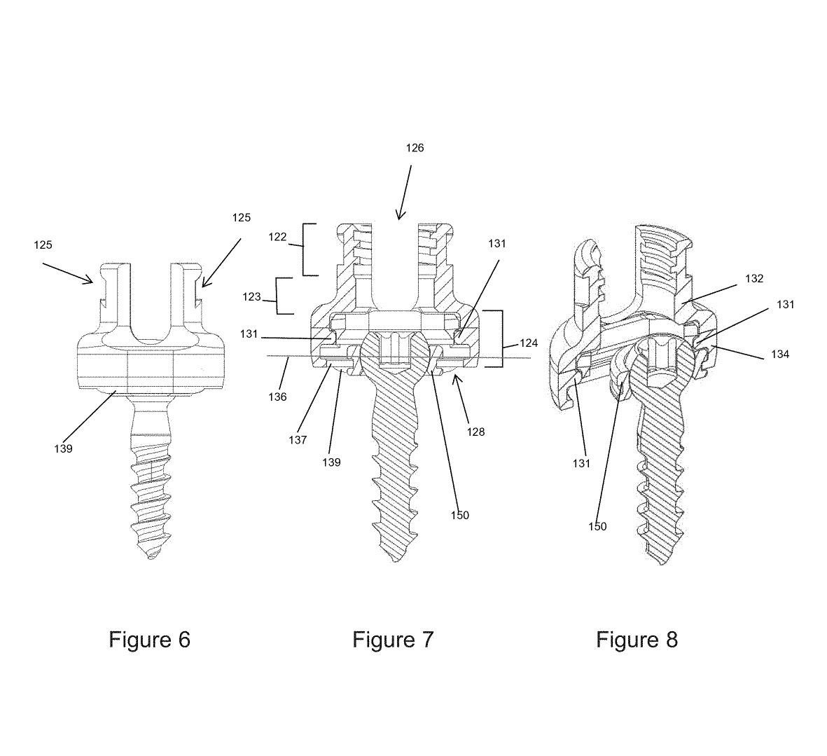

The lower component 134 can include a lip or rail 137 extending inwardly around a partial or entire perimeter of an inner surface of the lower component 134. In the illustrated embodiment, the lip or rail 137 is positioned proximate a bottom or distal end of the lower component 134. As shown in, for example, FIGS. 6-7 and 9-10, the sides of the lower component 134 include projections 139 extending downward or distally (e.g., extending downward or distally from the rail 137). The projections 139 have flat or straight (rather than curved) inner surfaces. In the illustrated embodiment, the lower component 134 includes protrusions 131 (shown in, for example, FIGS. 3, 7-8, and 10) extending inwardly from the ends of the lower component 134. The protrusions 131 can be proximate an upper or proximal end of the lower component 134 as shown.

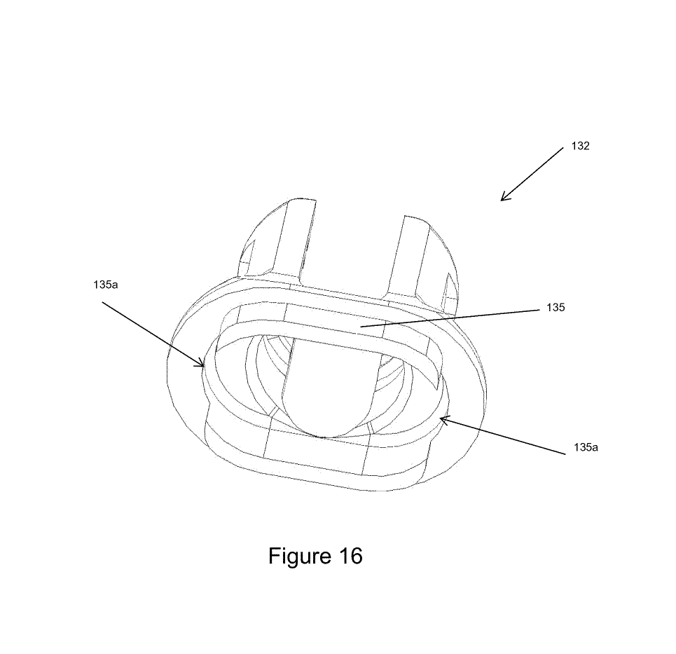

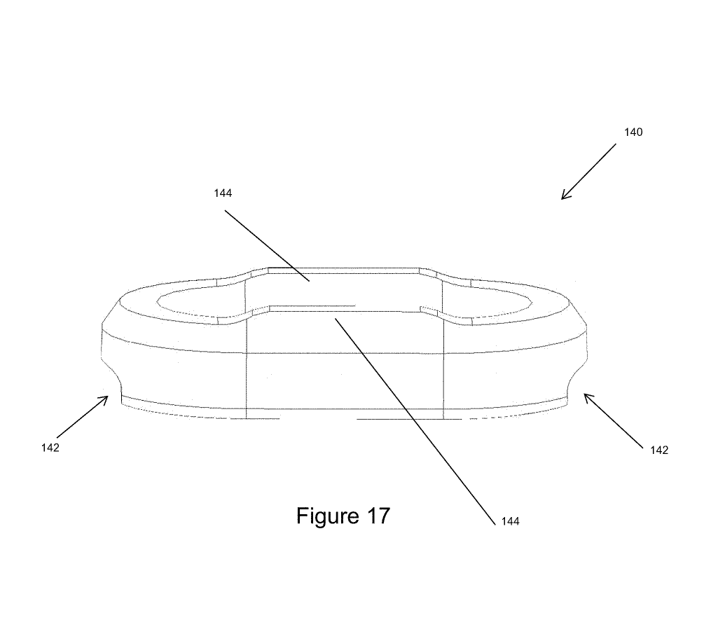

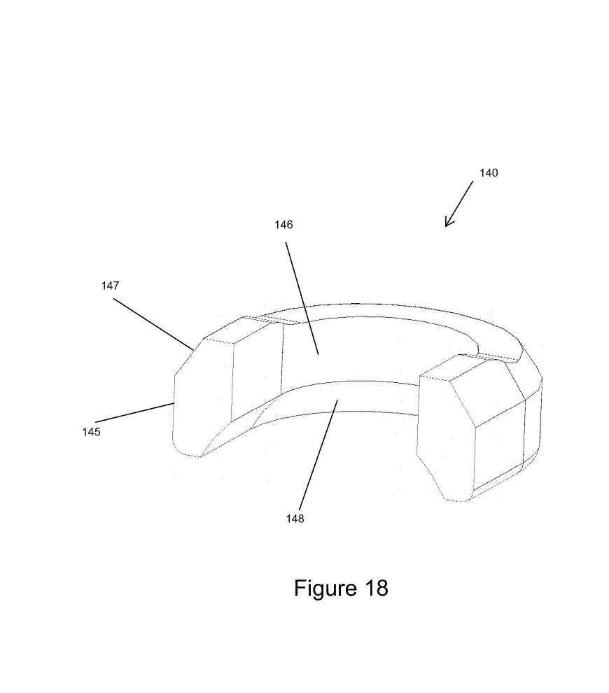

The load plate 140 has a generally oblong shape, as shown in, for example, FIGS. 3 and 17. The ends of the load plate 140 includes recesses 142 proximate or adjacent a bottom or distal end of the load plate 140. The sides of the load plate 140 can include raised portions 144 as shown in FIG. 17. As shown in the cross-sectional view of FIG. 18, an interior surface 146 of an upper portion of the load plate 140 can be flat or straight (e.g., parallel to first axis 127). An interior surface 148 of a lower portion of the load plate 140 can be tapered (e.g., downwardly outwardly tapered) or curved (e.g., inner-facing concave). In the illustrated embodiment, the interior surface 148 of the lower portion of the load plate 140 has a curvature that corresponds to the curvature of the head 112 of the screw 110. An outer surface of the load plate 140 can have a tapered (e.g., downwardly outwardly tapered) portion 147 adjacent a top or proximal end of the load plate 140. At least a portion 145 of the outer surface of the load plate 140 below or distal to the tapered portion 147 can be parallel to first axis 127.

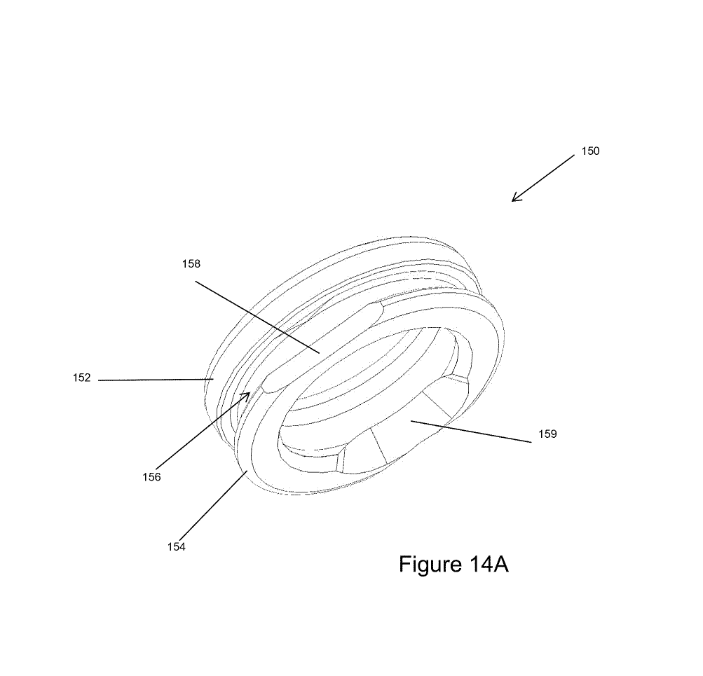

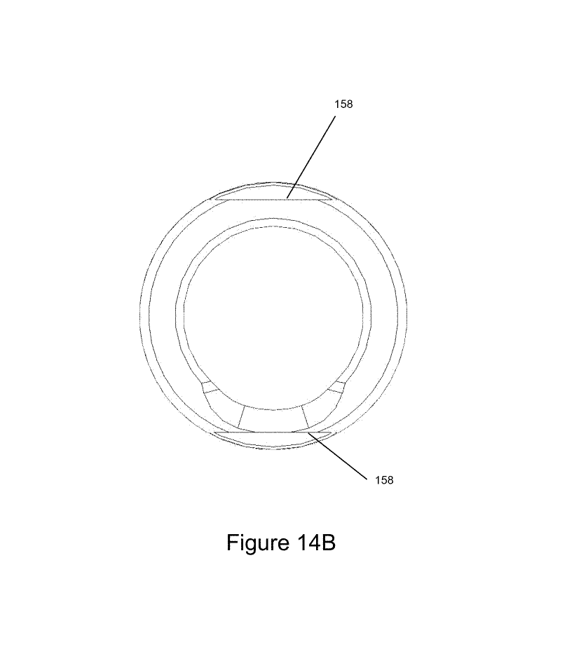

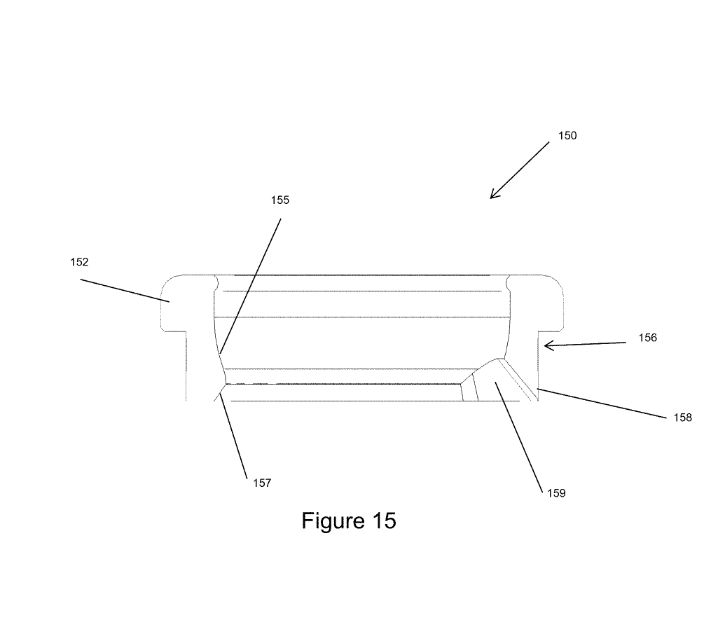

The collet 150, also shown in FIGS. 14A-15, is generally ring shaped. In the illustrated embodiment, the collet 150 includes an upper ring or extension 152 and a lower ring or extension 154. A recess 156 can be formed extending circumferentially around the collet 150 between the upper 152 and lower 154 rings. In the illustrated embodiment, an outer surface of the lower ring 154 includes two flattened or straight portions 158 on opposite sides of the lower ring 154. An inner surface 155 of the collet 150 can be curved. The curvature of the inner surface 155 can correspond to the curvature of the head 112 of the screw 110. A lower portion 157 of the inner surface of the collet 150 adjacent a bottom or distal end or edge of the collet 150 can be downwardly outwardly tapered as shown in FIG. 15. The inner surface of the lower ring 154 (and in some embodiments, a lower portion of an inner surface of the recess 156) can include an outwardly curved (or inwardly facing concave) section 159. In the illustrated embodiment, the curved section 159 is aligned with one of the flattened portions 158.

The screw 110, housing 120, load plate 140, and collet 150 can be preassembled. When assembled, the collet 150 is disposed around the head 112 of the screw 110. The upper 132 and lower 134 components of the housing 120 are coupled together as described herein. The load plate 140 is disposed within the housing 120 and may rest on or above the head 112 of the screw 110. Portion 145 of the exterior surface of the load plate 140 can be placed adjacent and may contact an inner surface of the flange 133 and/or lower extension 135 of the upper component 132 of the housing 120, as shown in FIG. 5. The gaps 135a in the lower extension 135 of the upper component 132 of the housing 120 can receive the ends of the load plate 140, for example, to orient or align the load plate 140 within the housing 120 and/or to help secure the load plate 140 within the housing 120 in the appropriate position. The recesses 142 in the ends of the load plate 140 can receive the protrusions 131 extending inwardly from the ends of the lower component 134 of the housing 120. In the illustrated embodiment, the raised portions 144 of the load plate 140 extend slightly above the rod seat for example as shown in FIG. 1. In use, the rod can contact the raised portions 144 of the load plate 140 when the rod is seated in the housing 120. The collet 150 is disposed within the housing 120, for example, within the lower component 134 of the housing 120 in the illustrated embodiment. The collet 150 can be top loaded into the housing 120. The collet 150 can be coupled to the screw 110 before being loaded into the housing 120. As shown, the recess 156 of the collet 150 can receive the rail 137 of the lower component 134 of the housing 120. The flattened portions 158 of the collet 150 are aligned with and contact the flat inner surfaces of the projections 139 of the lower component 134. The flattened portions 158 of the collet 158 can act as collet orientation features to help properly orient the collet 158 relative to the housing 120. In the illustrated embodiment, the collet 150 extends slightly below the lower component 134 of the housing 120. Portions of the screw head 112 can contact the interior surface 148 of the lower portion of load plate 140. The load plate 140 can act as an integrated moment cantilever as described in greater detail below.

In use, two or more preassembled screw assemblies 100 (with the screw 110, housing 120, load plate 140, and collet 150 preassembled) can be secured to two or more adjacent vertebrae, for example, in the pedicles of adjacent vertebrae, by threading the shaft 114 into the bone. A rod can then be placed in the third and fourth openings 160, 162 of the housings 120 to link the two or more screw assemblies 100. In some embodiments, the rod can be approximately straight. In other embodiments, the rod can be curved. The rod can be of various lengths and diameters. For example, the length can be selected based on the number of adjacent vertebrae the rod is intended to span. Once the rod is in place, set screws can be threaded into the upper portions 122 of the housings 120 to secure the rod and lock the housings 120 and rod in place in a chosen orientation. In some embodiments, one or more rods can be used to link one or more screw assemblies 100 with one or more other screw assemblies, for example, one or more screw assemblies 400 as described herein, and/or other components, for example, an occipital plate as described herein.

The translation screw assembly 100 advantageously allows for relative translation, angulation or pivoting, and/or rotation between the screw 110 and the housing 120 prior to being locked. Such translation, angulation or pivoting, and/or rotation can allow the housing 120 to be adjusted relative to the screw 110 when the screw 110 is secured in the patient's bone to allow the screw assembly 100 to accommodate or compensate for variations and deformities in the patient's spinal structure and allow two or more housings 120 to be sufficiently aligned such that the rod can be seated in and link the two or more housings 120.

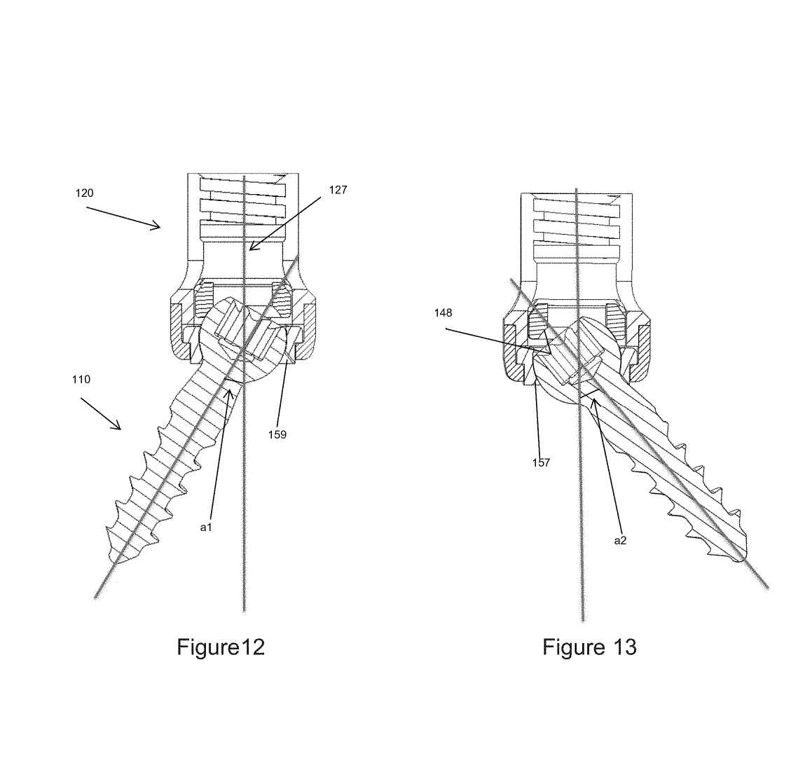

In the illustrated embodiment, the screw 110 can rotate relative to the collet 150 and housing 120 (i.e., can rotate about first axis 127 or an axis parallel to first axis 127 relative to the collet 150). The screw 110 can also pivot or angulate relative to the collet 150 and housing 120 as shown in FIGS. 12-13. In other words, the screw 110 can pivot or angulate relative to first axis 127. As shown, the tapered portion 157 of the inner surface of the collet 150 helps allow for angulation of the screw 110 relative to the collet 150 and accommodates a proximal end of the screw shaft 114 as the screw 110 is angled. As shown in FIG. 12, the screw assembly 110 allows for a range of angulation of the screw 110 relative to first axis 127 or an axis parallel to first axis 127 of up to angle al in any direction. In some embodiments, the range of angulation al is 30°. As shown in FIG. 13, the outwardly curved section 159 of the collet 150 allows for a greater range of biased angulation of up to angle a2 when the screw 110 (the screw shaft 114) is angled or pivoted toward the outwardly curved section 159. In some embodiments, the range of biased angulation a2 is 40°. The interior surface 148 of the lower portion of load plate 140 accommodates the screw head 112 and allows for angulation/pivoting of the screw 110 as also shown in FIGS. 12 and 13.

The screw 110 and collet 150 can also translate relative to the housing 120 along third axis 136. The flattened portions 158 of collet 150 can slide or translate along the flat inner surfaces of the projections 139 of the lower component 134 of the housing 120. In some embodiments, the screw 110 and collet 150 can translate 2mm in each direction of a neutral position or in either direction of the first axis 127. In some embodiments, the screw 110 and collet 150 can translate over a range of greater than or less than 2 mm in each direction. The contact between the flattened portions 158 of the collet 150 and the flat inner surfaces of the projections 139 of the lower component 134 prevents or inhibits the collet 150 from rotating (about first axis 127 or an axis parallel to first axis 127) relative to the housing 120.

Once the screw 110 is adjusted relative to the housing 120 and the rod is disposed in the housing 120 as desired or required, the set screw is threaded into the upper portion 122 of the housing 120 to secure the rod and lock the housing 120 and rod in place in the chosen orientation. The load plate 140 helps to prevent or inhibit further angulation or pivoting, rotation about first axis 127, and translation along axis 136 of the screw 110 relative to the housing 120 when the screw 110 is secured to the vertebral body and locked in position by the applied load of the set screw through the rod-load plate 140 interface. The load plate 140 transfers the load of the set screw to the screw head 112 by a load moment or cantilever created through two contact points on the load plate. A first contact point is created when the load plate 140 rests against at least one of the protrusions 131 of the lower component 134 of the housing 120. A second contact point is created between the load plate 140 and the screw head 112 itself. The locking function allowed by the load plate 140 advantageously allows the collet 150 to be separate from and move independently of the load plate 140 and housing 120.

### Friction Screw

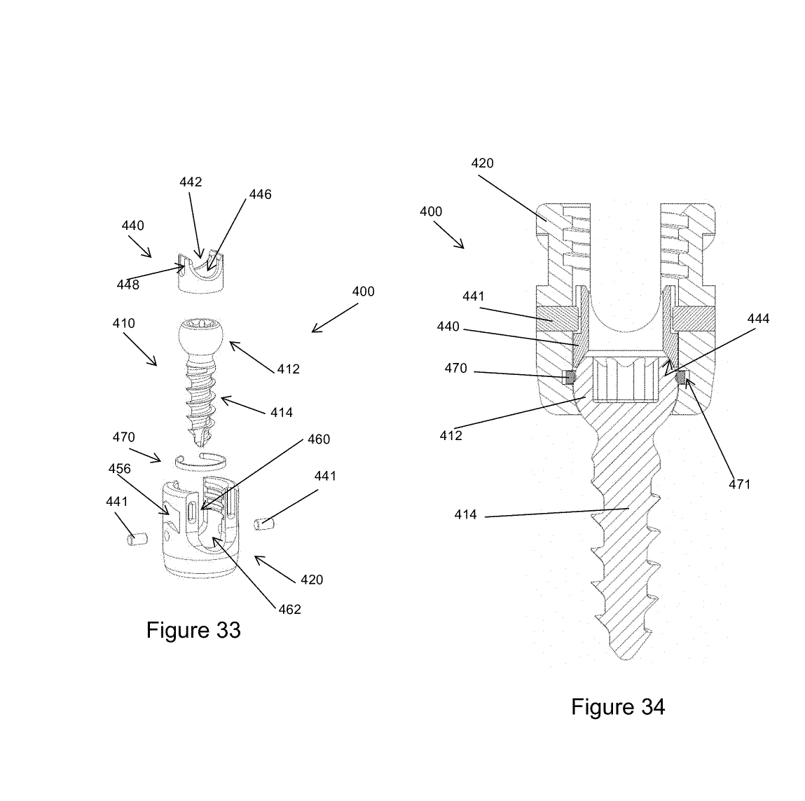

FIGS. 33-34 illustrate an example embodiment of a screw assembly 400. The screw assembly 400 in some embodiments may be used to attach to vertebrae on the posterior side of the cervical spine, e.g., to the pedicle, and may further be used to receive a rod that will connect to other screws or other devices attached to vertebrae of the cervical spine, the thoracic spine, and/or the occipital bone. Multiple screw assemblies 400 may be provided in use, for example, in adjacent vertebral bodies and on opposite sides of a patient's midline.

The screw assembly 400 can include a screw 410 configured to be secured to a vertebra, a housing 420, a saddle 440, and pins 441. The screw assembly 400 can further include a set screw and a spinal rod. In the illustrated embodiment, the screw assembly 400 also includes a c-clip ring 470. In some embodiments, one or more of the components of the screw assembly 400 is made of a metal, such as titanium or alloys thereof. For example, one or more components can be made at least partially of titanium 6AL 4V ELI. In some embodiments, the housing 420 is made of cobalt-chrome. In some embodiments, the spinal rod can be made of cobalt-chrome and/or titanium or a titanium alloy. In some embodiments, the screw assembly 400 is designed for use in the posterior cervical spine.

The screw 410 has an enlarged head 412 at a proximal end and a shaft or body portion 414 extending from the head 412 to a tip at a distal end. The head 412 can have a flattened proximal end or surface that can receive a screwdriver. The shaft 414 can be at least partially threaded and adapted to be implanted into a patient's spine, for example, into the pedicle of a vertebra.

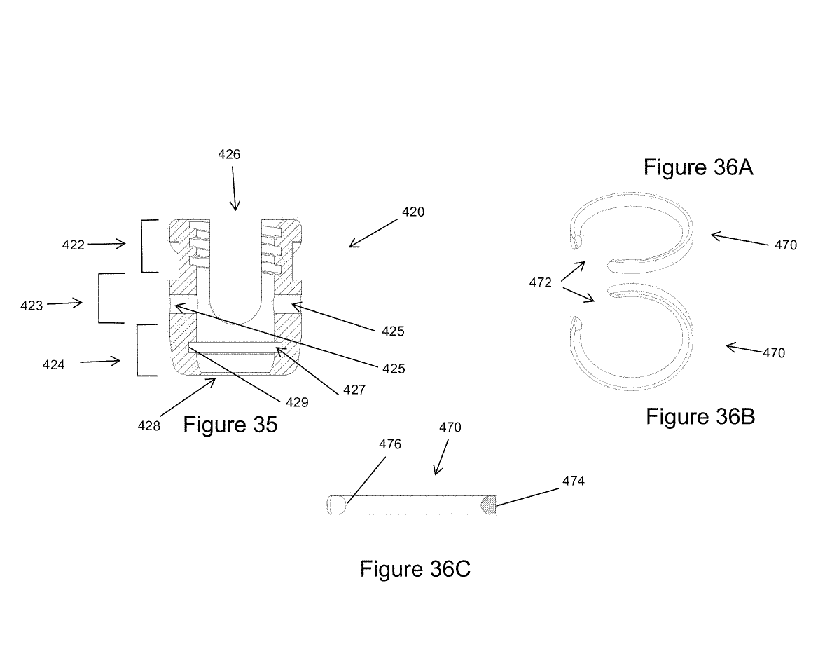

As shown in FIG. 35, the housing 420 includes an upper portion 422 having an upper opening 426, a lower portion 424 having a lower opening 428, and an intermediate portion 423. The upper opening 426 and lower opening 428 can extend along a first axis of the housing 420. The upper opening 426 and lower opening 428 can be connected so as to create a through hole passing from the upper opening 426, through the upper portion 426, intermediate portion 423, and lower portion 424, to the lower opening 428. In use, the screw 410 is disposed within the housing 420 such that the head 412 is within the lower portion 424 and the shaft 414 extends through the lower opening 428, as shown in FIG. 34.

A diameter of the upper opening 426 can be greater than a diameter of the lower opening 428. A diameter of the enlarged head 412 of the screw 410 can be smaller than the diameter of the upper opening 426. The screw 410 can therefore be loaded into the housing 420 from the upper opening 426. In the illustrated embodiment, a diameter of the lower opening 428 of the housing 420 is less than a greatest diameter of the enlarged head 412 of the screw 410. The head 412 can therefore be prevented or inhibited from passing through the lower opening 428. As shown in FIG. 34, when assembled, a portion of the enlarged head 412 of the screw 410 contacts at least a portion of an inner wall of the lower portion 424 of the housing 420.

In the illustrated embodiment, an interior of the upper portion 422 is generally cylindrical. An interior surface of the lower portion 424, or at least a lower section of the lower portion 424, can have a gradually decreasing diameter towards the bottom of the housing 420. The interior surface of the lower portion 424 or lower section of the lower portion 424 can be conical, tapered, or curved. In the illustrated embodiment, an interior surface of an upper section of the lower portion 424 is generally cylindrical, and the interior surface of the lower section of the lower portion 424 is curved.

The housing 420 further includes a third opening 460 and a fourth opening 462 extending along a second axis of the housing 420 that is transverse to the first axis. The third opening 460 and fourth opening 462 intersect an upper edge of the housing 420 and separate the upper portion 422 and intermediate portion 423 of the housing 420 into two opposing arms. In the illustrated embodiment, the third opening 460 and fourth opening 462 are generally U-shaped, although other shapes are also possible. In use, the third opening 460 and fourth opening 462 receive the rod such that the rod is disposed within the intermediate portion 423, and lower or distal portions of the third opening 460 and fourth opening 462 define a seat for the rod.

In the illustrated embodiment, the upper portion 422 of the housing 420 is internally threaded to receive and engage an externally threaded set screw. The threading may not extend below a point at or below the rod when the rod is disposed in the housing 420 in use. In other embodiments, the upper portion 422 may be externally threaded to receive and engage an internally threaded set screw or cap, or the upper portion 422 may receive and engage a closure mechanism via means other than threading. The set screw can have square or modified square threads, although other types of threads are also possible.

The intermediate portion 423 can include one or more holes 425 extending perpendicularly to the first axis and second axis. In the illustrated embodiment, the intermediate portion 423 includes two holes 425 positioned opposite each other with one hole 425 through each of the arms of the housing 420. An outer surface of the housing 420 can include one or more indentations 456 that receive an insertion tool during use.

In the illustrated embodiment, the lower portion 424 of the housing 420 includes an undercut 427 or recess or channel in the inner wall of the lower portion 424 of the housing 420. The undercut 427 can extend around an entire circumference of the inner wall of the lower portion of the housing 420.

As shown in FIGS. 33-34, the saddle 440 can have a generally cylindrical outer surface. An upper surface of the saddle 440 has an indentation 442 sized and shaped to receive the rod in use. The indentation 442 can be shaped approximately as a portion of a cylinder. As shown, the indentation 442 can cause an upper portion of the saddle 440 to be generally U-shaped with two opposing projections or arms. A lower surface of the saddle 440 has an indentation 444 sized and shaped to receive the enlarged head 412 of the screw 410. An outer surface of the saddle 440 can include one or more indentations 448. In the illustrated embodiment, the outer surface of the saddle 440 includes two indentations 448, one in each of the arms of the upper portion of the saddle 440 such that the indentations 448 are positioned opposite each other. Each of the indentations 448 can receive a pin during assembly as described in greater detail herein. The saddle 440 also includes a through hole 446 that allows a screwdriver to reach the proximal end of the head 412 in use. Although in the illustrated embodiment the saddle 440 is a unitary piece, in other embodiments, the saddle 440 can have two or more separate parts.

As shown in FIGS. 36A-36C, the c-clip ring 470 is a generally circular or c-shaped (when viewed from the top) ring having a slit or gap 472. In the illustrated embodiment, an outer wall or surface 474 of the c-clip ring 470 is flat or straight and an inner wall or surface 476 is curved inward-facing convex. The c-clip ring 470 is generally sized and shaped to be at least partially disposed within the undercut 427 of the housing 420 as shown in FIG. 34. In the illustrated embodiment, an outer diameter of the c-clip ring 470 is less than a diameter of the undercut 427. In other words, when the c-clip ring 470 is disposed in the undercut 427, there is a gap 471 between the outer surface 474 of the c-clip ring 470 and an outer wall 429 of the undercut 427. As also shown in FIG. 34, the c-clip 470 is generally sized and/or designed such that the inner wall or surface 476 contacts the enlarged head 412 of the screw 410 in use. In some embodiments, an inner diameter of the c-clip ring 470 is less than a diameter of at least a portion of the enlarged head 412 of the screw 410 such that in a resting or default state, the inner surface 476 of the c-clip ring 470 contacts and is relatively snug about the enlarged head 412 of the screw 410.

In some embodiments, the slit or gap 472 allows the c-clip ring 470 to flex and expand to be placed around the enlarged head 412 of the screw 410 during assembly. In some embodiments, the slit or gap 472 allows the c-clip ring 470 to collapse to be inserted through an upper opening 426 of the housing 420 during assembly. The c-clip ring 470 is advanced downward into the housing 420 until the c-clip ring 470 reaches the undercut 427. The c-clip ring 470 can then spring open (or revert to its original size and/or shape) into the undercut 427. In use, contact between the inner surface 476 of the c-clip ring 470 and the enlarged head 412 of the screw 410 creates a friction force, which can provide resistance to relative movement between the screw 410 and the housing 420.

The screw 410, housing 420, saddle 440, and c-clip ring 470 can be preassembled. The c-clip ring 470 can be loaded into the upper opening 426 and pushed downward until the c-clip ring 470 reaches and springs open into the undercut 427. The screw 410 can be loaded into the upper opening 426 and pushed or pulled down into the housing 420 until the enlarged head 412 contacts and is seated against the inner wall of the lower portion of the housing 420. The c-clip ring 470 can flex or expand outward into the gap 471 to allow a portion of the enlarged head 412 of the screw 410 to pass by the c-clip ring 470 and be seated in the housing 420. The saddle 440 can then be loaded into the housing 420 such that the indentation 442 is aligned with the third and fourth openings 460, 462 of the housing 420 and the indentations 448 are aligned with the holes 425 in the housing 420. Pins 441 are press-fit into the holes 425 and indentations 448 to secure the screw 410, housing 420, c-clip ring 470, and saddle 440 together.

When assembled, the screw 412 can rotate and pivot polyaxially with respect to the housing 420. In some embodiments, the pins 441 provide a downward force on the saddle 440, which then presses downward on the head 412 of the screw 410. This creates friction between the head 412 of the screw 410 and the lower portion of the housing 420. The screw 410 therefore generally does not rotate or pivot relative to the housing 420 unless the friction force is overcome, for example, by the surgeon or other user physically moving the screw 410 or housing 420 relative to the other. In some embodiments, contact and/or friction between the head 412 of the screw 410 and the inner surface 476 of the c-clip ring 470 also or alternatively resists relative movement between the screw 410 and the housing 420. The curved inner surface 476 of the c-clip ring 470 can advantageously allow for a smooth transition or movement between the head 412 of the screw 410 and the c-clip ring 470 as the screw assembly 400 is assembled and/or during relative rotation or pivoting between the head 412 of the screw 410 and the housing 420.

In use, two or more screw assemblies 400 can be secured to two or more adjacent vertebrae, for example, in the pedicles of adjacent vertebrae, by threading the shaft 414 into the bone. A rod can then be placed in the third and fourth openings 460, 462 of the housings 420 and on the saddles 440 to link the two or more screw assemblies 400. In some embodiments, the rod can be approximately straight. In other embodiments, the rod can be curved. The rod can be of various lengths and diameters. For example, the length can be selected based on the number of adjacent vertebrae the rod is intended to span. Once the rod is in place, set screws can be threaded into the upper portions 422 of the housings 420 to secure the rod and lock the housings 420 and rod in place in a chosen orientation. In some embodiments, one or more rods can be used to link one or more screw assemblies 400 with one or more other screw assemblies, for example, one or more screw assemblies 100 as described herein, and/or other components, for example, an occipital plate as described herein.

### Occipital Plate

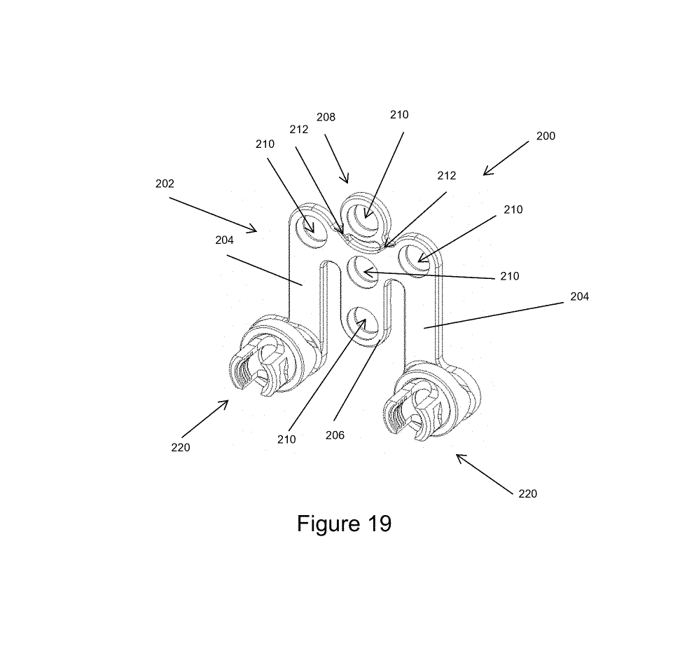

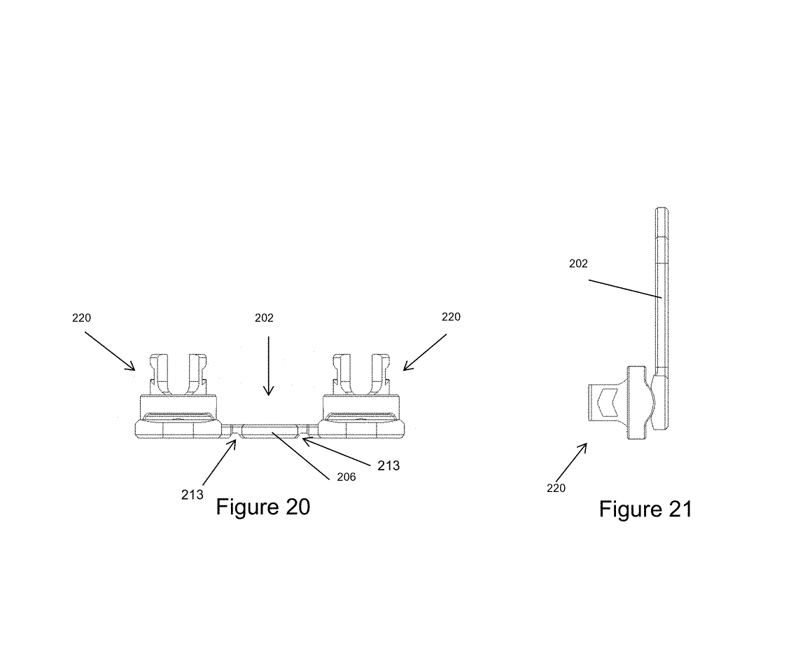

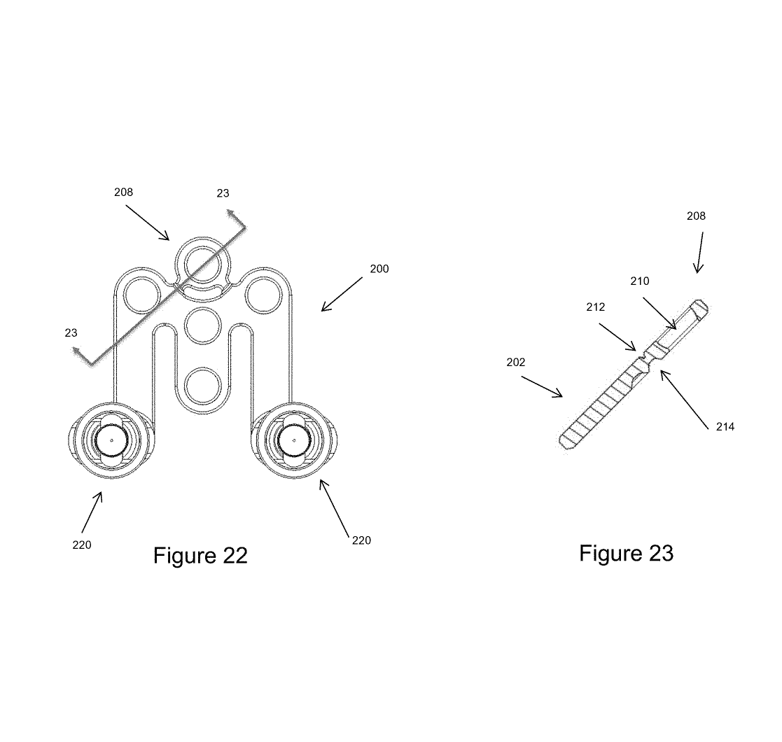

FIGS. 19-27C illustrate an example embodiment of an occipital plate assembly 200. As shown, the occipital plate assembly 200 includes a plate body 202 and at least one rod-receiving assembly 220. In the illustrated embodiment, the plate body 202 has two lateral arms 204 and a central arm 206 positioned between the two lateral arms 204 such that the plate body 202 is generally “M”-shaped. In use, the lateral 204 and central 206 arms extend in a direction, when positioned against a patient's occipital bone, generally parallel to a line extending from a center of a base of the occipital bone to the sagittal suture. In other words, the central arm 206 can lie within the sagittal plane in use. For convenience, the lateral 202 and central 206 arms are described as extending in a superior-inferior direction herein. As shown, the lateral arms 204 can be longer than the central arm 206, and the lateral arms 202 can extend superiorly and inferiorly to the central arm 206. In the illustrated embodiment, the plate body 202 includes a tab 208 aligned with and superior to the central arm 206 and extending superiorly of superior ends of the lateral arms 204.

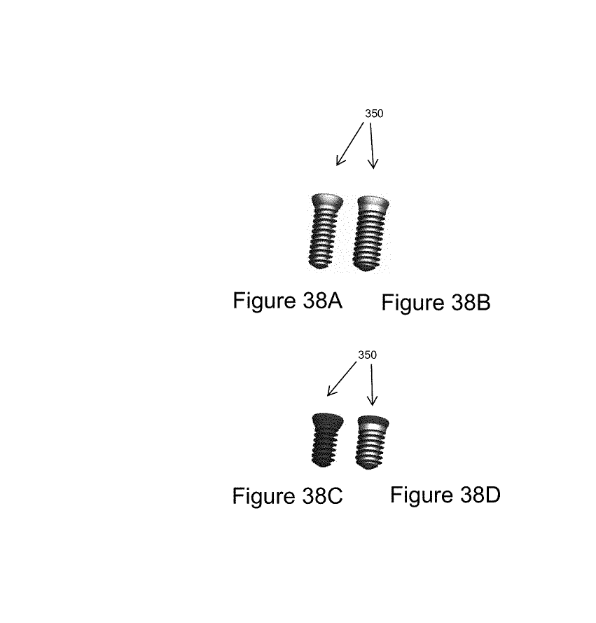

The plate body 202 includes one or more screw receiving holes 210. In the illustrated embodiment, a screw receiving hole 210 is positioned proximate the superior end of each lateral arm 204, two screw receiving holes 210 are positioned in the central arm 206 and aligned along a superior-inferior axis with respect to each other, and a screw receiving hole 210 is also positioned in the tab 208. In use, bone screws, such as occipital screws 350 shown in FIGS. 38A-38D, can be inserted through one or more of the screw receiving holes 210 and into the patient's bone(s) to secure the occipital plate assembly 200 to the patient's bone(s). In some embodiments, the occipital screws 350 can be inserted through the screw receiving holes 210 into bone over a range of insertion angulation of ±10°. In some embodiments, the occipital plate assembly 200 is secured to the patient's skull, for example, the occipital bone, in use.

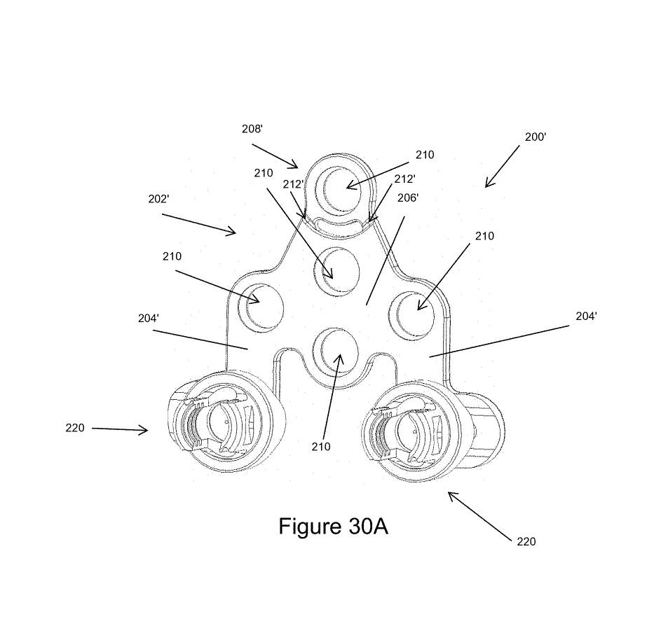

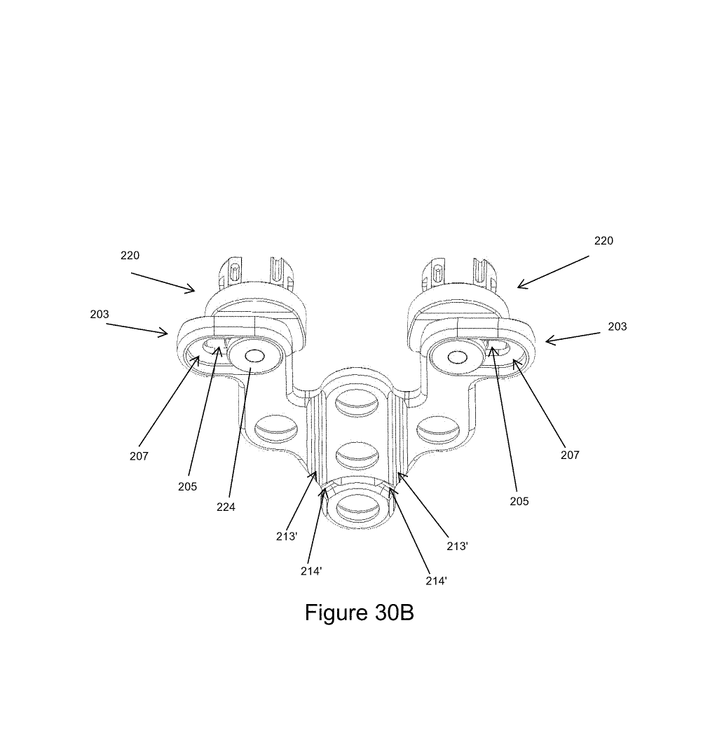

Other configurations and arrangements of the plate body 202 and screw receiving holes 210 are also possible. For example, FIGS. 30A-30B illustrate an alternative embodiment of an occipital plate assembly 200′ that includes a plate body 202′ and rod receiving assemblies 220. The plate body 202′ has two lateral arms 204′, a central portion 206′, and a tab 208′ extending from and/or coupled to a superior end of the central portion 206′. The plate body 202′ includes screw receiving holes 210 proximate the superior ends of each of the lateral arms 204′, two screw receiving holes 210 in the central portion 206′ that are aligned with each other along a superior-inferior axis, and a screw receiving hole 210 in the tab 208′. In the embodiment of FIG. 30A, the screw receiving holes 210 in the lateral arms 204′ and central portion 206′ are arranged to form corners of a diamond shape. In the illustrated embodiment, the rod receiving assemblies 220 are located at or proximate inferior ends of the lateral arms 204.

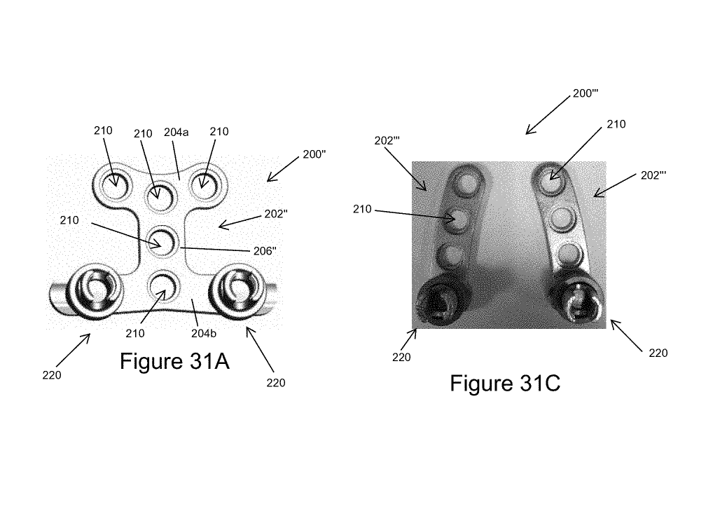

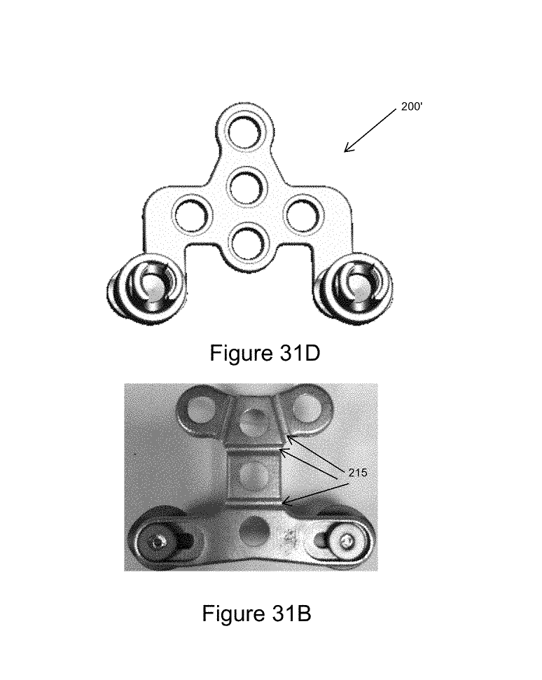

As another example, FIGS. 31A-31B illustrate another alternative embodiment of an occipital plate assembly 200″ that includes a plate body 202″ and rod receiving assemblies 220. The plate body 202″ is I-shaped or generally I-shaped and has an upper arm 204a, a lower arm 204b, and a central arm 206″ extending between the upper arm 204a and lower arm 204b. The upper arm 204a can have a V-shaped or an upward (or superior when attached to the body) facing concave profile as shown. In other embodiments, the upper arm 204a can be straight. The plate body 202″ includes screw receiving holes 210 proximate the lateral ends of the upper arm 204a, a screw receiving hole 210 at or proximate a center of the upper arm 204a, a screw receiving hole 210 in the central arm 206″, and a screw receiving hole 210 at or proximate a center of the lower arm 204b. The screw receiving holes 210 in the center of the upper arm 204a, in the central arm 206″, and in the center of the lower arm 204b can be aligned along a superior-inferior axis. As shown, the screw receiving holes 210 can be arranged to form a Y or T shape. The rod receiving assemblies 220 can be located at or proximate lateral ends of the lower arm 204b as shown.

In some embodiments, instead of a single and/or unitary plate body, an occipital plate assembly 200″′ can include two plates 202″′ as shown in FIG. 31C. In use, the plates 202″′ can be positioned bilaterally on the patient's skull, with one of the plates 202″′ disposed on each side of the midline of the skull. In the illustrated embodiment, the plates 202″′ have a slightly curved or arcuate profile. Each plate 202″′ includes a rod receiving assembly 220 at or proximate one end of the plate 202″′ configured to be positioned inferiorly in use. In the illustrated embodiment, each plate 202″′ includes three screw receiving holes 210 aligned along a curved or arcuate longitudinal axis of the plates 202″′, although more or fewer screw receiving holes 210 are also possible. The occipital plate assemblies 200′, 200″, 200″′ can include some or all of the features shown and described herein with respect to occipital plate assembly 200.

In some embodiments, the tab 208 is arranged to overlie or be placed over or proximate the patient's occipital protuberance in use such that a screw inserted through the screw receiving hole 210 in the tab 208 can be secured to the occipital protuberance. The occipital plate assembly 200 can include one or more reliefs 212, 214 at or along junctions between the tab 208 and the plate body 202. At the reliefs 212, 214, the occipital plate assembly 200 is thinner. The reliefs 212, 214 allow the tab 208 to be bent upward and/or downward (posteriorly or away from the patient and/or anteriorly or toward the patient in use) relative to the plate body 202. As shown in FIG. 23, in some embodiments, a top surface of the occipital plate assembly 200 includes a sharp (or relatively short or narrow) relief 212. In some embodiments, a bottom surface of the occipital plate assembly 200 includes a generous (or relatively long or wide) relief 214. The sharp relief 212 allows the material to shear if the tab 208 is excessively bent downward such that the tab 208 can be removed from the plate body 202 if the tab 208 is not desired or required. The generous relief 214 allows the tab 208 to bend upward relative to the plate body 202 without shearing to allow the tab 208 to be adjusted to accommodate the particular patient's anatomy. In some embodiments, the occipital plate assembly does not include a relief in the top surface of the occipital plate assembly between the tab and the plate body, for example, as shown in the example embodiment of FIG. 31D. In such embodiments, the occipital plate assembly may or may not include a relief in the lower surface of the occipital plate assembly between the tab and the plate body.

In some embodiments, the plate body 202, 202′ includes one or more reliefs 213, 213′ in the bottom surface of the plate body 202, 202′ at, along, and/or proximate junctions between the lateral arms 204, 204′ and central arm 206 or central portion 206′, for example, as shown in FIGS. 20, 25A-25C, 30B. As shown in FIG. 31B, the occipital plate assembly 200″ can include one or more reliefs 215 in the lower surface of the plate body 202″. The occipital plate assembly 200″ can include one or more reliefs 215 between a central portion of the upper arm 204a and lateral portions of the upper arm 204a (e.g. between the screw receiving hole 210 in the center of the upper arm 204a and the screw receiving holes 210 proximate the lateral ends of the upper arm 204a), at, along, and/or proximate a junction between the upper arm 204a and the center arm 206″, at, along, and/or proximate a junction between the lower arm 204b and the center arm 206″, and/or between a central portion of the lower arm 204b and lateral portions of the lower arm 204b (e.g., between the screw receiving hole 210 in the center of the lower arm 204b and the rod receiving assemblies 220). As shown, each relief can extend along a longitudinal axis that is transverse to an axis connecting the midpoints of the screw receiving holes 210 nearest to and on either side of the relief. These reliefs 213, 213′, 215 can advantageously allow portions of the plate body 202, 202′, 202″ to be bent upward and/or downward (posteriorly or away from the patient and/or anteriorly or toward the patient in use) relative to other portions of the plate body 202, 202′, 202″ to allow the plate body 202, 202′, 202″ to be contoured to the patient's anatomy.

In the illustrated embodiment, the occipital plate assembly 200 includes a rod-receiving assembly 220 coupled to the plate body 202 proximate the inferior end of each of the lateral arms 204. The plate body 202 includes a mounting section 203 at the inferior end of each of the lateral arms 204. As shown, the mounting section 203 is oblong, and a longitudinal axis of the mounting section 203 extends in a medial-lateral direction perpendicular to the lateral arms 204. The occipital plate body 202′ can similarly include a mounting section 203 at the inferior end of each of the lateral arms 204′. A longitudinal axis of the mounting section 203 can extend in a medial-lateral direction perpendicular to the lateral arms 204′. The occipital plate body 202″ can include a mounting section 203 at or proximate each of the lateral ends of the lower arm 204b. A longitudinal axis of the mounting section 203 can extend in a medial-lateral direction parallel or generally parallel to the lower arm 204b. In some embodiments, each of the plates 202″′ can include a mounting section 203 at or proximate the end of the plate 202″′ configured to be positioned inferiorly in use. A longitudinal axis of the mounting section 203 can extend in a medial-lateral direction generally perpendicular to the plate 202′″.

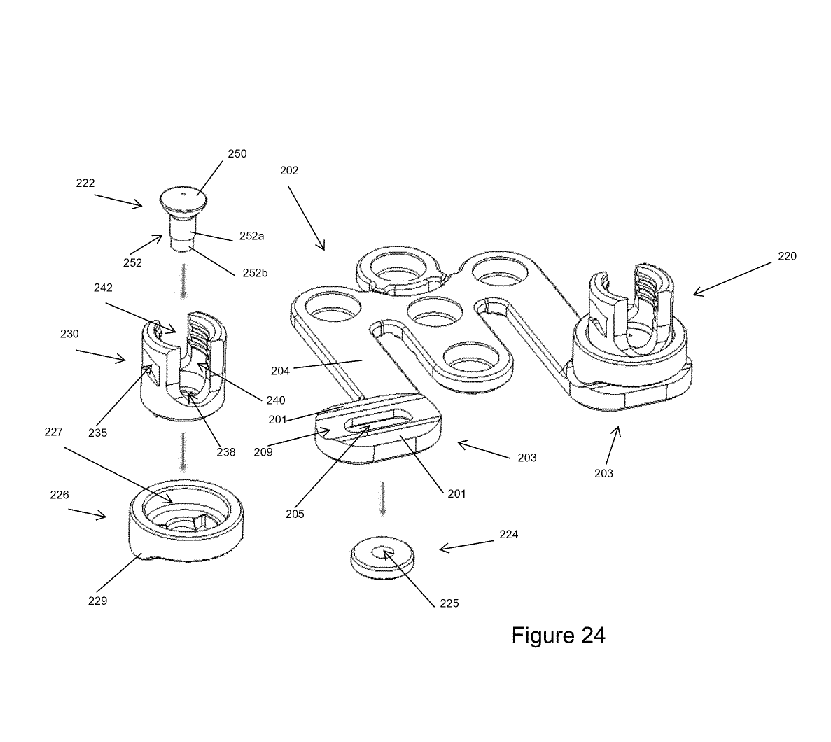

The mounting section 203 includes a slot 205 that extends through the plate 200 from a top surface to a bottom surface and that extends along a length of the mounting section 203 along the longitudinal axis of the mounting section 203. A top surface of the mounting section 203 can include a channel 209 surrounding the slot 205 and extending across a length of the mounting section 203. In the illustrated embodiment, the channel 209 extends across the entire length of the mounting section 203. A bottom surface of the mounting section 203 can include an elongated recess 207 surrounding the slot 205, for example as shown in the embodiment of FIG. 30B. In the illustrated embodiment, superior and inferior portions 201 (shown in FIG. 24) of the mounting portions 203 are tapered downward and outward to superior and inferior edges of the mounting portions 203.

As shown in, for example, the exploded view of FIG. 24, the rod-receiving assembly 220 includes a pivot post 222, a retaining washer 224, a clamp plate 226, and a housing 230. The pivot post 222 has an enlarged head 250 and a shaft 252. The shaft can have an upper shaft portion 252a and a lower shaft portion 252b having a smaller diameter than the upper shaft portion 252a. The retaining washer 224 has a through-hole 225. The through-hole 225 is sized to receive the lower shaft portion 252b of the pivot post 222. In the illustrated embodiment, the retaining washer 224 is circular. The retaining washer 224 is sized to be received in the recess 207 of the mounting section 203.

A top of the clamp plate 226, also shown in FIGS. 28A-28C, includes a recess 227 as shown in FIG. 24. The recess 227 is sized and shaped to receive a portion of the housing 230. A floor of the recess 227 includes an angulation limitation opening or slot 228. As shown, the angulation limitation slot 228 can be bowtie-shaped. The angulation limitation slot 228 can have a circular central portion 228a and a fan shaped portion 228b extending outwardly from each side of the central portion 228a. The clamp plate 226 also includes a protruding ridge 229 extending downward from a bottom surface of the clamp plate 226. The ridge 229 extends across an entire diameter of the clamp plate 226, and the slot 228 is positioned through the ridge 229. In the illustrated embodiment, the clamp plate 226 is circular (e.g., when viewed from the top).

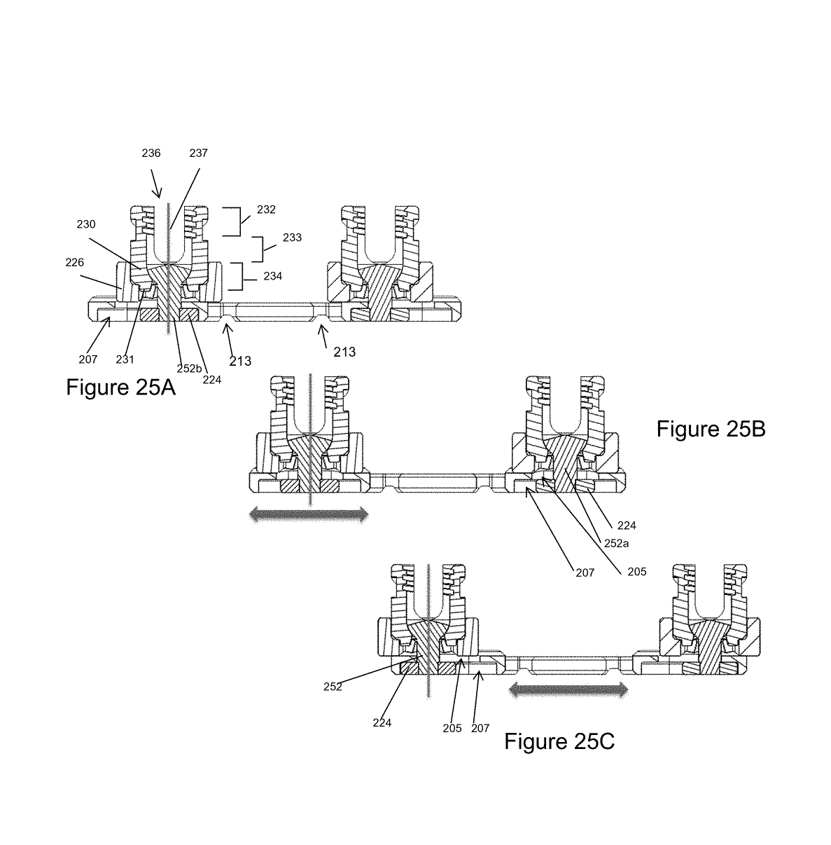

As shown in, for example, FIGS. 24-25A, the housing 230 includes an upper portion 232 having an upper opening 236, a lower portion 234 having a lower opening 238, and an intermediate portion 233. The upper opening 236 and lower opening 238 can extend along a first axis 237 of the housing 230. The upper opening 236 and lower opening 238 can be connected so as to create a through hole passing from the upper opening 236, through the upper portion 236, intermediate portion 233, and lower portion 234, to the lower opening 238. In use, the pivot post 222 is disposed within the housing 230 such that the head 250 is within the lower portion 234 and the shaft 252 extends through the lower opening 238. A lower surface of the head 250 of the pivot post 222 can be tapered or rounded or curved. An inner surface of the lower portion 234 of the housing 230 can be tapered or rounded or curve to correspond to the tapered or rounded or curved lower surface of the head 250. A diameter of the lower opening 238 can be smaller than a diameter of the upper opening 126. A diameter of the enlarged head 250 of the pivot post 222 can be smaller than the diameter of the upper opening 236 and the diameter of the lower opening 238. As shown in FIGS. 29A-29C, the housing 230 includes two angulation tabs 231 protruding from a bottom surface of the housing 230 and extending from opposite sides of the lower opening 238.

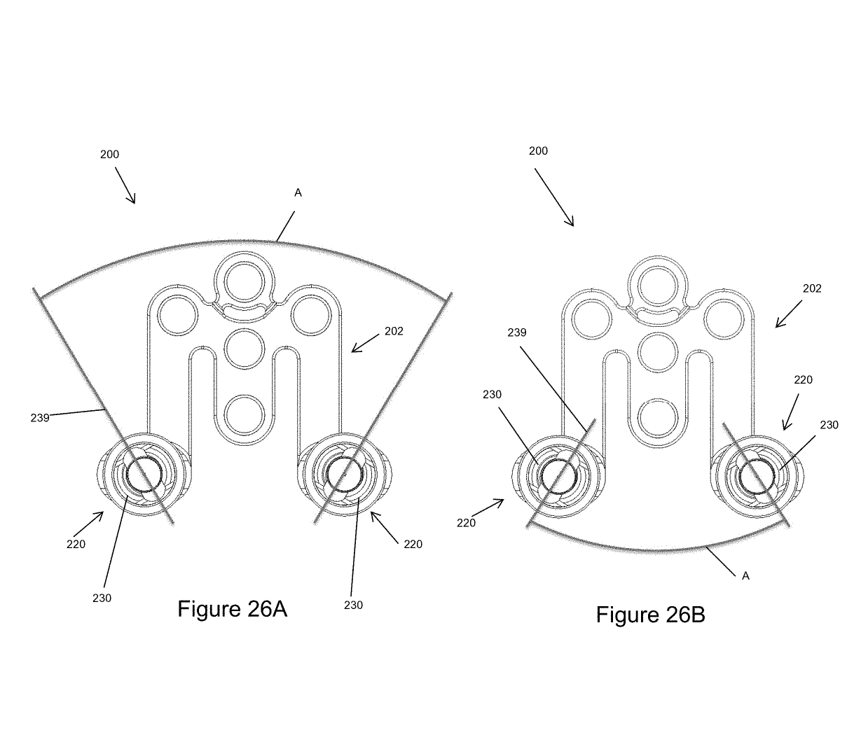

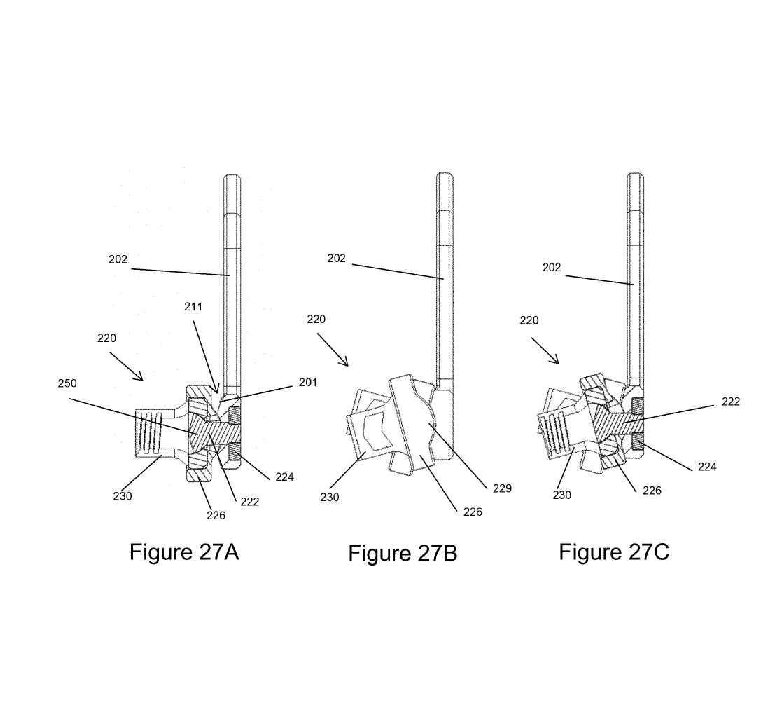

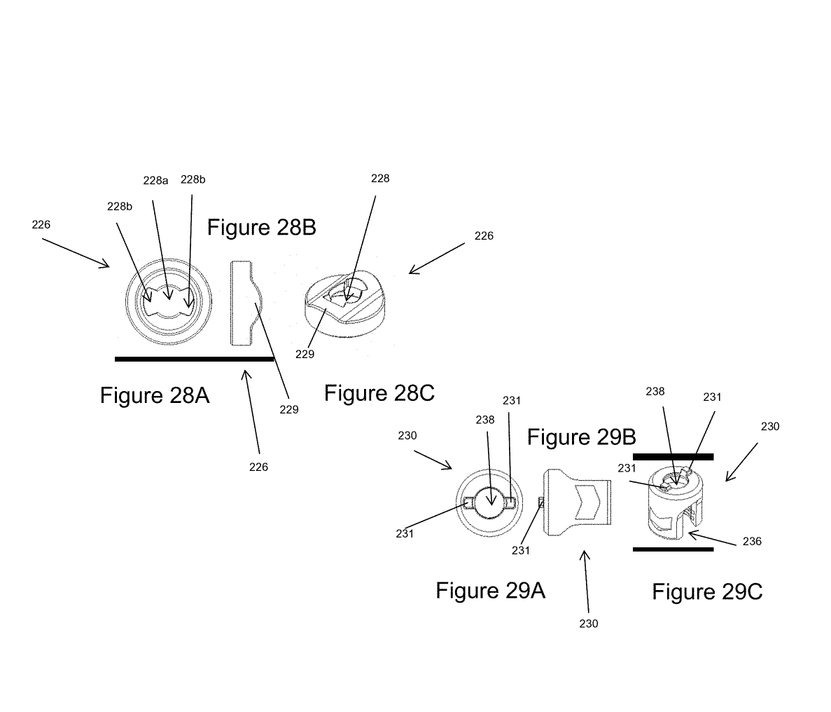

The housing 230 further includes a third opening 240 and a fourth opening 242 extending along a second axis 239 (shown in FIGS. 26A-26B) of the housing 230 that is transverse to the first axis 237. In the illustrated embodiment, a line connecting the two angulation tabs 231 extends perpendicular to the second axis 239. The third opening 240 and fourth opening 242 intersect an upper edge of the housing 230 and separate the upper portion 232 and intermediate portion 233 of the housing 230 into two opposing arms. In the illustrated embodiment, the third opening 240 and fourth opening 242 are generally U-shaped, although other shapes are also possible. In use, the third opening 240 and fourth opening 242 receive the rod such that the rod is disposed within the intermediate portion 233, and lower or distal portions of the third opening 240 and fourth opening 242 define a seat for the rod.

In the illustrated embodiment, an interior of the upper portion 232 is generally cylindrical. An exterior of the upper portion 232 can also be generally cylindrical. In other embodiments, the exterior of the upper portion 232 can have a squared or slightly squared shape. In the illustrated embodiment, the upper portion 232 of the housing 230 is internally threaded to receive and engage an externally threaded set screw. The threading may not extend below a point at or below the rod when the rod is disposed in the housing 230 in use. In other embodiments, the upper portion 232 may be externally threaded to receive and engage an internally threaded set screw or cap, or the upper portion 232 may receive and engage a closure mechanism via means other than threading. The set screw can have square or modified square threads, although other types of threads are also possible. An outer surface of the housing 230 can include one or more indentations 235 that receive an insertion tool during use.

The pivot post 222, retainer washer 224, clamp plate 226, and housing 230 can be preassembled with the plate body 202. When assembled, the clamp plate 226 is positioned on top of the mounting portion 203 such that the ridge 229 is disposed at least partially in the channel 209. The ridge 229 and channel 209 can act as alignment features to help properly align the rod-receiving assembly 200 on the plate body 202. In some embodiments, there is a gap 211 (shown in FIG. 27A) between bottom surfaces of the clamp plate 226 on either side of the ridge 229 and top surfaces of the mounting portion 203 on either side of the channel 209. The lower portion 234 of the housing 230 is disposed within the recess 227 of the clamp plate 226 such that the angulation tabs 231 protrude into the angulation limitation slot 228. The head 250 of the pivot post 222 is disposed within the lower portion 234 of the housing 230, and the shaft 252 extends through the lower opening 238, the central portion 228a of the angulation limitation slot 228, and the slot 205 of the mounting section 203. The retainer washer 224 is disposed in the recess 207 in the bottom surface of the mounting section 203, and the lower shaft portion 252b of the pivot post 222 is received in the through-hole 225 of the retainer washer 224 and secured to the retainer washer 224 to secure the rod-receiving assembly 220 together and to the plate body 202. The pivot post 222 can be securely attached to the retainer washer 224 via, for example, a welded, threaded, or pinned joint or connection.

In use, a rod can then be placed in the third and fourth openings 240, 242 of the housing 230, and a set screw can be threaded into the upper portion 232 of the housing 230 to secure the rod and lock the housing 230 and rod in place. The rod-receiving assembly 220 can be locked by a locking force created by a downward force of the set screw being transmitted through the rod to a top surface of the clamp plate 226 and opposing forces of an interface between the lower opening 238 of the housing 230 and head 250 of the pivot post 222 and an interface between the lower shaft portion 252b of the pivot post 222 and the retaining washer 224 at or along the recess 207 of the mounting portion 203.

In some embodiments, the rod can link the occipital plate assembly 200, 200′, 200″, 200″′ to one or more bone screw assemblies 310, such as pedicle screw assemblies, secured to one or more of the patient's vertebrae, for example, to one or more pedicles, for example, as shown in FIG. 32B. In some embodiments, one or more translation screw assemblies 100 and/or screw assemblies 400 as described herein can be used in combination with the occipital plate assembly 200, 200′, 200″, 200″′, and a rod can link a rod-receiving assembly 220 to one or more translation screw assemblies 100 and/or screw assemblies 400.

The rod-receiving assemblies 220 advantageously allow for relative medial-lateral translation, medial-lateral angulation, and/or cranial-caudal (superior-inferior) angulation between the rod-receiving assemblies 220 or portions thereof (e.g., the housings 230) and plate body 202. Such translation and/or angulation can allow the rod receiving assemblies 220 to be adjusted relative to the plate body 202 when the plate body 202 is secured to the patient's bone to allow the rod receiving assemblies 220 to accommodate or compensate for variations and deformities in the patient's spinal structure and can allow for the rod receiving assemblies 220 to be sufficiently aligned with bone screw assemblies that may be secured to one or more of the patient's vertebrae so that a rod can be seated in and link the rod receiving assemblies 220 and bone screw assemblies.

As shown in FIGS. 25A-25C, the retainer washer 224 can slide or translate in the recess 207 in the bottom surface of the mounting portion 203, the shaft 252 (e.g., upper shaft portion 252a) of the pivot post 222 can slide or translate within the slot 205 of the mounting portion 203, and the ridge 229 of the clamp plate 226 can slide or translate along the channel 209 to allow the rod receiving assemblies 220 to translate in a medial-lateral direction relative to the plate body 202. FIG. 25B illustrates a neutral positon in which the rod receiving assemblies 220 are centered on the mounting portions 203. FIG. 25A illustrates a position in which the two rod receiving assemblies 220 are closest together, and FIG. 25C illustrates a position in which the two rod receiving assemblies 220 are farthest apart. Each rod receiving assembly 220 can be translated independently of each other and can be positioned at any point between an inclusive of a most medial position (as shown in FIG. 25A) and a most lateral position (as shown in FIG. 25C). Therefore, for example, one rod receiving assembly 220 may be positioned in a most medial position while the other is positioned in a most lateral position. In some embodiments, each rod receiving assembly 220 has a range of motion of 2.5 mm in each direction of the neutral position. Each rod receiving assembly 220 therefore has a total range of motion of 5 mm, and the two rod receiving assemblies 220 can therefore have a total combined range of motion of 10 mm. Larger or smaller ranges of motion are also possible.

The angulation tabs 231 of the housing 230 can pivot or angulate within the fan shaped portions 228b of the slot 228 in the clamp plate 226 to allow for medial-lateral angulation of the housings 230 relative to the clamp plate 226 and/or plate body 202, as shown in FIGS. 26A-26B. In a neutral position, the second axis 239 of the housing 230 extends in a super-inferior direction. The housing 230 can pivot or angulate relative to the plate body 202 such that superior ends of the second axis 239 move laterally as shown in FIG. 26A or medially as shown in FIG. 26B. In some embodiments, the second axis 239 of each housing 230 can move 30° in both directions such that combined the two housings 230 can angulate over a range (indicated by arc A in FIGS. 26A-26B) over 60°. Greater or smaller ranges of medial-lateral angulation are also possible.

As shown in FIG. 27A-27C, the housings 230 and clamp plates 226 can pivot or angulate in a cranial-caudal or superior-inferior direction relative to the plate body 202, pivot post 222, and retainer washer 224. The ridge 229 of the clamp plate 226 and channel 209 of the mounting portion 203 can be rounded to allow the ridge 229 to rock within the channel 209 and allow for the cranial-caudal angulation movement. The gap 211 and/or tapered portions 201 of the mounting portion 203 can accommodate cranial-caudal angulation of the clamp plate 226. The corresponding tapered or rounded or curved surfaces of the lower surface of the pivot post head 250 and inner surface of the lower portion 234 of the housing 230 allow the head 250 to slide along the inner surface of the lower portion 234 and allow the housing 230 to pivot or angulate relative to the pivot post 222. In some embodiments, the housings 230 (e.g., the first axis 237) can pivot or angulate up to 15° from a neutral positon (in which the first axis 237 extends perpendicular to the plate body 202) in each of the cranial/superior and caudal/inferior directions such that the housing 230 has a total range of cranial-caudal angulation motion of 30°. Smaller or larger ranges of angulation are also possible. The housing 230 can be secured in a desired orientation or angulation when the rod is disposed in the housing 230 and a force is applied by the set screw to the rod and housing 230 to create a locking force as described above.

### Rods and System

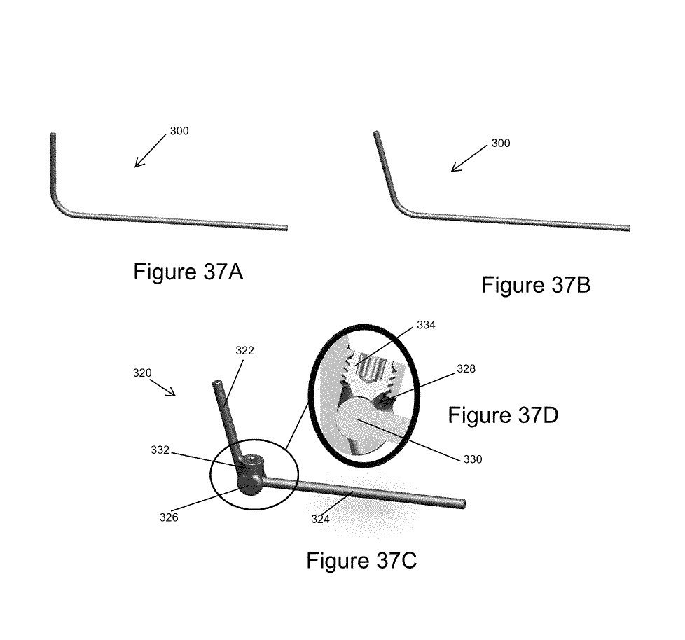

As described herein, in some embodiments, one or more rods can connect an occipital plate with one or more screws, for example, an occipital plate as described herein with one or more translation screw assemblies 100, friction screw assemblies 400, and/or other screws, in use. The rods can be made of, for example, titanium or alloys thereof, Cobalt-Chromium (CoCr), and/or any other implantable grade material. FIGS. 37A and 37B illustrate example embodiments of pre-lordosed rods 300. The pre-lordosed rods 300 can be provided having various degrees of curvature or angulation, for example, 90° as shown in FIG. 37A and/or 75° as shown in FIG. 37B.

FIGS. 37C-37D illustrate an example embodiment of an adjustable hinged rod 320. The rod 320 has a first portion 322 and a second portion 324. A first end 326 of the first portion 322 has a cavity 328 that receives an enlarged head 330 at a first end of the second portion 324. A protrusion 332 extends from the first end 326 of the first portion 322. The protrusion 332 is internally threaded to receive a set screw 334. When the set screw 334 is loosened or removed, the head 330 can be pivoted or rotated within the cavity 328 to adjust an angle between the first portion 322 and second portion 324. When the desired or required angle is achieved, the set screw 334 is tightened to maintain the angle. When tightened, the set screw 334 can contact the head 330 and can secure the head 330 against a wall of the cavity 328.

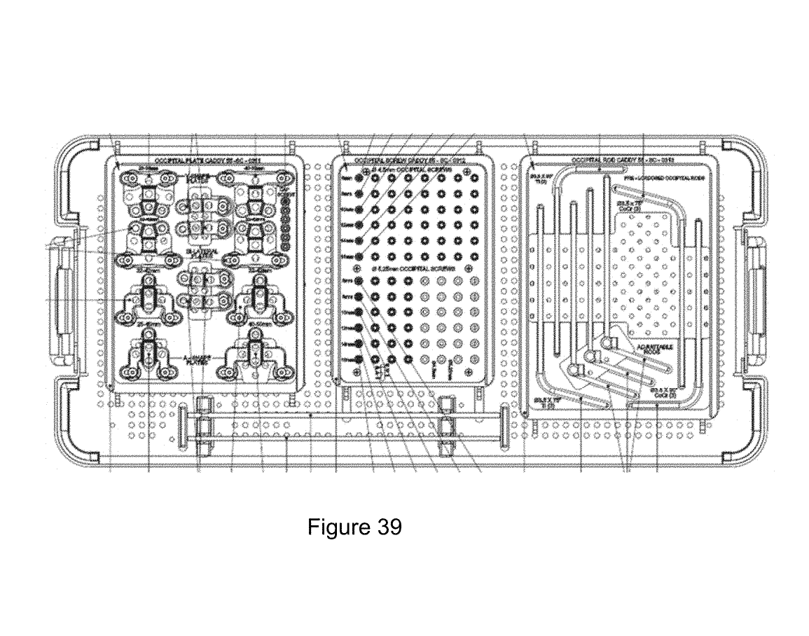

In some embodiments, a kit can include one or more occipital plate assemblies, such as one or more occipital plate assemblies 200, 200′, 200″, 200″′ described herein, one or more occipital screws 350, and/or one or more rods, for example, one or more curved rods 300 and/or hinged rods 320. An example embodiment of a kit or implant tray is shown in FIG. 39. The kit can include occipital screws 350 having a variety of different lengths (e.g., 6-16 mm, in some embodiments in 2 mm increments) and/or diameters (e.g., 4.5 mm and 5.25 mm), as shown in FIGS. 38A-38D. The kit can include rods of different lengths (e.g., 50 mm×125 mm; 125 mm×125 mm). The rods can have a diameter of 3.5 mm or about 3.5 mm. The kit can include curved rods 300 having different degrees of curvature, for example, such as the rods 300 shown in FIGS. 37A and 37B.

In some embodiments, the occipital plate assembly 200, 200′, 200″ can be provided in various sizes. For example, a small size occipital plate assembly 200 can be sized such that when the two rod receiving assemblies 220 are closest together, the rod receiving assemblies 220 are 25 mm apart, and when the rod receiving assemblies 220 are farthest apart, the rod receiving assemblies are 35 mm apart. A medium size occipital plate assembly 200 can be sized such that when the two rod receiving assemblies 220 are closest together, the rod receiving assemblies 220 are 32 mm apart, and when the rod receiving assemblies 220 are farthest apart, the rod receiving assemblies are 42 mm apart. A large size occipital plate assembly 200 can be sized such that when the two rod receiving assemblies 220 are closest together, the rod receiving assemblies 220 are 40 mm apart, and when the rod receiving assemblies 220 are farthest apart, the rod receiving assemblies are 50 mm apart. Other sizes and dimensions are also possible. In some embodiments, a kit can include occipital plate assemblies of different configurations and/or sizes.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A translation screw assembly comprising:
a screw having a threaded shaft and an enlarged head at a proximal end; and
a housing having an upper portion with an upper opening and a lower portion with a lower opening extending along a first axis of the housing, wherein the enlarged head of the screw is disposed within the housing and the shaft extends out of the housing through the lower opening, the housing having a third opening and a fourth opening along a second axis transverse to the first axis adapted to receive an elongated rod, wherein the screw is configured to translate, pivot, and rotate relative to the housing.

2. The translation screw assembly of claim 1 further comprising a generally circular collet disposed around the enlarged head of the screw and disposed adjacent the lower opening of the housing, wherein the screw is configured to rotate and pivot relative to the collet.

3. The translation screw assembly of claim 2 wherein a lower portion of the housing is oblong and inner surfaces of sides of the lower portion of the housing adjacent the lower opening are straight, wherein an outer surface of the collet comprises two opposing straight portions, and wherein the straight portions of the outer surface of the collet are configured to contact and translate along the straight inner surfaces of the sides of the lower portion of the housing such that the collet and screw can translate relative to the housing and the collet is rotationally fixed relative to the housing.

4. The translation screw assembly of claim 1 further comprising a load plate disposed within the housing, wherein at least a portion of the screw head is configured to contact an inner surface of a lower portion of the load plate.

5. The translation screw assembly of claim 1 wherein the housing comprises an upper component and a lower component, the upper component comprising the upper portion and the upper opening and the lower component comprising the lower portion and the lower opening, wherein the upper component and lower component are fixedly coupled together.

6. An occipital plate assembly comprising:
a plate body comprising at least one screw receiving hole configured to receive a bone screw therethrough; and
an occipital protuberance tab coupled to the plate body via at least one junction, wherein a top surface of the at least one junction comprises a material relief comprising a reduced thickness area configured such that if a sufficient downward force is applied to the occipital protuberance tab, the occipital protuberance tab shears off from the plate body at the at least one junction.

7. The occipital plate assembly of claim 6, wherein a bottom surface of the at least one junction comprises a second material relief comprising a reduced thickness area configured such that the occipital protuberance tab can be bent upwards relative to the plate body.

8. The occipital plate assembly of claim 7, wherein the second material relief is wider than the material relief.

9. The occipital plate assembly of claim 6, wherein occipital protuberance tab comprises a screw receiving hole configured to receive a bone screw therethrough.

10. The occipital plate assembly of claim 6, further comprising at least one rod receiving assembly configured to receive a spinal rod.

11. An occipital plate assembly comprising:
a plate body comprising at least one screw receiving hole configured to receive a bone screw therethrough; and
at least one rod receiving assembly coupled to the plate body and comprising a housing configured to receive an elongated rod, wherein the housing is configured to translate in a medial-lateral direction, angulate in a medial-lateral direction, and angulate in a cranial-caudal direction relative to the plate body.

12. The occipital plate assembly of claim 11,
the rod receiving assembly further comprising a pivot post, a clamp plate, and a retaining washer;
wherein the housing has an upper portion with an upper opening and a lower portion with a lower opening extending along a first axis of the housing, wherein an enlarged head of the pivot post is disposed within the housing and a shaft of the pivot post extends out of the housing through the lower opening, the housing having a third opening and a fourth opening along a second axis transverse to the first axis adapted to receive the elongated rod;
wherein the plate body comprises a mounting portion having a first slot therethrough;
wherein the clamp plate comprises a recess and a second slot therethrough;
wherein the housing is partially seated in the recess of the clamp plate and the clamp plate is disposed on the mounting portion; and
wherein the shaft of the pivot post extends through the second slot of the clamp plate and the first slot of the plate body and is secured by the retaining washer disposed adjacent a bottom surface of the mounting portion.

13. The occipital plate assembly of claim 12, wherein the housing, pivot post, clamp plate, and retaining washer are configured to translate along the mounting portion.

14. The occipital plate assembly of claim 12, wherein the second slot of the clamp plate comprises a central portion through which the shaft of the pivot post extends and two side portions extends from opposites sides of the central portion, wherein the housing comprises two tabs protruding from a bottom surface of the housing on opposite sides of the lower opening, and wherein the tabs extend into the side portions of the second slot of the clamp plate and are configured to angulate relative to the clamp plate within the side portions of the second slot.

15. The occipital plate assembly of claim 12, wherein a bottom surface of the clamp plate comprises a curved ridge, a top surface of the mounting portion comprises a curved channel, and the ridge is at least partially disposed in the channel, and wherein the ridge is configured to rock within the channel to allow for cranial-caudal angulation of the housing relative to the plate body.

16. An occipital fixation system, comprising:
an occipital plate assembly comprising:
a plate body sized for positioning on a patient's occipital bone, the plate body comprising at least one screw receiving hole configured to receive a bone screw therethrough; and
a pair of rod receiving assemblies coupled to the plate body each comprising a housing configured to receive an elongated rod, wherein each housing is configured to translate in a medial-lateral direction, angulate in a medial-lateral direction, and angulate in a cranial-caudal direction relative to the plate body;
a plurality of screw assemblies configured to be inserted into adjacent vertebrae of the patient's cervical spine, each of the plurality of screw assemblies comprising:
a screw having a threaded shaft and an enlarged head at a proximal end; and
a housing having an upper portion with an upper opening and a lower portion with a lower opening extending along a first axis of the housing, wherein the enlarged head of the screw is disposed within the housing and the shaft extends out of the housing through the lower opening, the housing having a third opening and a fourth opening along a second axis transverse to the first axis adapted to receive an elongated rod, wherein the screw is configured to translate, pivot, and rotate relative to the housing; and
a pair of rods sized to be received within the rod receiving assemblies of the occipital plate assembly and the housings of the screw assemblies to connect the occipital plate assembly when positioned on the patient's occipital bone to the screw assemblies when inserted into adjacent vertebrae of the patient's cervical spine.
